(19) United States
(12) Patent Application Publication
(10) Pub. No.: US 2008/0226609 A1
(43) Pub. Date: Sep. 18, 2008

(54) Transplantation of Glial Restricted Precursor-Derived Astrocytes for Promotion of Axon Growth

(75) Inventors: Chris Proschel, Pittsford, NY (US); Margot Mayer-Proschel, Pittsford, NY (US); Jeannette Davies, Denver, CO (US); Stephen Davies, Denver, CO (US); Mark Noble, Brighton, NY (US)

Correspondence Address: FISH & RICHARDSON P.C., P.O BOX 1022, Minneapolis, MN, 55440-1022, US

(73) Assignees: UNIVERSITY OF ROCHESTER, Rochester, NY (US); BAYLOR COLLEGE OF MEDICINE, HOUSTON, TX (US)

(21) Appl. No.: 12/064,778

(22) PCT Filed: Aug. 28, 2006
(86) PCT No.: PCT/US2006/033757

§ 371 Date: Apr. 21, 2008

Related U.S. Application Data
(60) Provisional application No. 60/711,498, filed on Aug. 26, 2005.
(60) Provisional application No. 60/712,044, filed on Aug. 29, 2005.

(51) Int. Cl. C12N 5/06; A61K 35/30; A61P 25/28

(52) U.S. Cl. 424/93.7; 435/325

(57) ABSTRACT

Provided herein are compositions and methods for the treatment of central nervous system (CNS) lesions comprising administration of glial restricted precursor (GRP) derived astrocytes (GDAs).

### CROSS REFERENCE TO RELATED APPLICATIONS

This application claims priority to U.S. Ser. No. 60/711,498 filed Aug. 26, 2005 and U.S. Ser. No. 60/712,044 filed Aug. 29, 2005.

### STATEMENT REGARDING FEDERALLY SPONSORED RESEARCH

This invention was made with government support under Grants RO1 NS04642 and RO1 NS42820 awarded by the National Institutes of Health. The government has certain rights in the invention

### BACKGROUND

Traumatic injury to the adult central nervous system (CNS) is associated with multiple types of damage, all of which pose substantial challenges to tissue repair. Promoting regenerative growth of severed motor and sensory axons requires the provision of appropriate substrates and/or the overriding of a variety of inhibitors that prevent axon regeneration. The expression of molecular inhibitors of axon growth has been extensively characterized in both glial scar tissue (Bundesen, et al., J. Neurosci. 23:7789-800 2003; De Winter, et al., Exp. Neurol. 175:61-75 2002; Moreau-Fauvarque, et al., J. Neurosci. 23:9229-39 2003; Tang, et al., J. Neurosci. Res. 71:427-44 2003) and in CNS myelin (Chen, et al., Nature 403:434-9 2000; McKerracher, et al., Neuron 13:805-11 1994; Wang, et al., Nature 417:941-4 2002). Injury is also associated with physical disruption of normal tissue structure and with the generation of disorganized scar tissue (Berry, et al., Acta Neurochir. Suppl. 32:31-53 1983; Windle, et al., J. Comp. Neurol. 96:359-69 1952) that lacks the linear organization of adult CNS white matter thought to be required for long distance axon growth (Davies, et al., J. Neurosci. 19:5810-22 1999; Davies, et al., Nature 390:680-3 1997; Davies, et al., J. Neurosci. 14:1596-1612 1994; Pettigrew, et al., J. Neurosci. 19:8358-66 1999).

A variety of approaches have been used to promote regenerative growth of both sensory and motor axons, with a particular focus on the transplantation of a variety of cell types, often in combination with other therapies. Cell based transplantation strategies for promoting axon growth across spinal cord injuries (Reier, et al., Neurorx. 1:424-51 2004) have included the use of Schwann cells, olfactory ensheathing cells, neural stem cells, neonatal brain astrocytes and bone-marrow derived cells. Cellular transplant strategies have also been combined with delivery of neurotrophic factors, bioactive substances that override or degrade the scar, and with the use of biomaterials to offer both potential substrates and organized tissue structures resulting in varying degrees of successful axon regeneration (Bunge, et al., Neuroscientist 7:325-39 2001). However, transplantation of various cell types directly into or adjacent to traumatic spinal cord injuries has not resulted in the regeneration of significant numbers of endogenous axons across the site of injury (Han, et al., Glia 45:1-16 2004; Hill, et al., Exp. Neurol. 190:289-310 2004; Hofstetter, et al., Nat. Neurosci. 8:346-53 2005; McDonald, et al., Nat. Med. 5:1410-12 1999).

### SUMMARY

Provided herein are compositions and methods for the treatment of spinal cord injury or other traumatic or degenerative disorders of the central nervous system (CNS), promotion of axon regeneration, suppression of astrogliosis, re-alignment of host tissues, and the delay of axon growth inhibitory proteoglycans expression. Thus, provided is a method of treating a CNS lesion in a subject, comprising administering to the lesion a composition comprising glial restricted precursor (GRP) derived astrocytes (GDAs).

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B and 1C show dorsal column (FIGS. 1A and 1B) and dorsolateral funiculus (FIG. 1C) lesion models. FIG. 1A shows a horizontal view of a cervical spinal cord. The right side dorsal column white matter was lesioned at the C1/C2 spinal level and GDAs were transplanted (♦) directly into lesion centers and within the rostral and caudal lesion margins. FIG. 1B shows a sagittal view of cervical spinal cord. The ability of either biotinylated dextran amine (BDA) labeled endogenous axons or axons from microtransplanted adult sensory neurons (DRGs) expressing Green Fluorescent Protein (GFP) to cross GDA transplanted lesions was assayed. The shaded box in FIG. 1A or the trapezoid in FIG. 1B represents the lesion site. Cf; cuneate fasciculus; Gf, gracile fasciculus; CST, corticospinal tract; CC, central canal; gm, gray matter. FIG. 1C shows the right side dorsolateral funiculus that was lesioned at the C3/C4 spinal level. GDAs were transplanted as described for dorsal column lesions. To trace axotomized rubrospinal tract axons, BDA was injected into the left side red nucleus (RN) 8 days prior to experimental endpoints.

FIG. 2 is a graph showing quantification of numbers of regenerating BDA+ axons in GDA transplanted versus control dorsal column white matter at 8 days post injury/transplantation. BDA labeled axons were counted in every third sagittally oriented section within the lesion center, and at points 0.5 mm, 1.5 mm and 5 mm rostral to the injury site, up to and including the dorsal column nuclei. Note that 61% of BDA+ axons had reached the centers of GDA transplanted lesions and 39% to 0.5 mm beyond injury sites, compared to just 4% (lesion center) and 3.8% (0.5 mm rostral) present in controls. The total absence of axons at 5.0 mm rostral and DCN in controls and the steady decline in numbers of BDA+ axons within rostral white matter is indicative of staggered axon growth beyond sites of injury in GDA transplanted groups. Increases in numbers of BDA+ axons in GDA treated animals compared to controls were statistically significant (p<0.01) in all of rostral spinal cord regions. Error bars+/−one standard deviation. dcn, dorsal column nuclei.

FIG. 3 shows endogenous sensory axon regeneration across GDA-transplanted dorsal column injuries at 8 days post lesion/transplantation. Panel (a) is a montaged, low magnification confocal image scanned from a single 25 μm thick sagittal section showing BDA labeled sensory axons that have entered, grown within and exited a hPAP+/GDA− transplanted dorsal column lesion. LC, lesion center. Panel (b) is a high magnification image of a rostral graft/host interface showing BDA+ axons exiting the GDA graft and entering host white matter. A few axons were observed to have turned away from the interface and grown back towards the lesion center (arrowhead). Panel (c) shows that in control lesions, the vast majority of BDA+ axons have failed to leave caudal lesion margins, denoted by hypertrophic GFAP+ astrocytes, and have formed dystrophic endings. Panel (d) is a high magnification image showing numerous BDA+ axons that have successfully crossed the host/graft interface at the caudal lesion margin. A few cut axons (arrowheads) failed to leave the caudal lesion interface and exhibit turning and/or dystrophic endings, particularly in regions containing few hPAP+ GDAs. Panel (e) shows BDA+ axons located near the pial surface and ventral regions of cuneate white matter at 1.5 mm rostral to a GDA bridged lesion site. Panel (f) shows BDA+ axon growth cones in white matter 1.5 mm rostral to the lesion site often display streamlined growth cones indicative of rapid growth. Scale bars: a, c, 100 μm; b, d, e, 50 μm; f, 5 μm and 10 μm.

FIG. 4 shows a comparison of GDA versus GRP transplants to promote axon growth across dorsal column injuries from adjacent microtransplanted adult sensory neurons at 8 days post lesion/transplantation. Panel (a) is a montaged, confocal image scanned from a single 75 μm thick sagittal oriented section showing GFP+ axons entering and exiting a dorsal column lesion bridged with hPAP+ GDAs. Panel (b) shows that in 2 cases in which GDA transplants did not adequately fill the injury site or migrate into lesion margins, GFP+ sensory axons failed to cross the caudal lesion margin and instead formed dystrophic endings identical to those in control untreated injuries. LC, lesion center. Panel (c) is a confocal montage showing the complete failure of transplanted GRPs to support the growth of GFP axons across a dorsal column injury. Note that, despite the ability of transplanted GRPs to span the injury site, the majority of GFP+ axons have formed dystrophic endings within the caudal lesion margin. Scale bars: a, 300 μm; b, 100 μm; c, 200 μm.

FIG. 5A shows GDA reorganization of lesion margins. Control lesions at 4 days (panel (a)) and particularly at 8 days post injury (panel (c)) display a dense mesh-work of hypertrophic cell bodies and processes of endogenous astrocytes within lesion margins that is typical of forming glial scar tissue. Panel (b) shows that at 4 days post injury/transplantation, “flares” of hPAP+ GDAs are interwoven with re-aligned host GFAP+ astrocytes within lesion margins (caudal margin shown). Processes of both transplanted GDAs and host astrocytes are oriented toward the lesion center. Note that hPAP+ GDAs are not GFAP+. Panel (d) shows that at 8 days post injury/transplantation, GDAs have effected a reduction in host astrogliosis and a striking realignment of host GFAP+ astrocytes compared to control panel (c).

FIG. 5B is a graph showing quantification of alignment of host GFAP+ processes in lesion margins. The angles measured between each pair of GFAP+ processes in control (n=100) and GDA transplanted lesion margins (n=100) are graphically displayed in a histogram. Each bin along the x axis represents degrees of difference in angle between a pair of processes. 0° is parallel, 90° is perpendicular. The y axis indicates the number of GFAP+ pairs of processes within each bin. Note the striking difference in alignment of GFAP+ host astrocytic processes in margins of GDA transplanted lesions versus controls. GDA transplanted lesions have an average angle of just 11.6° (median 7°) between paired processes, versus 59.4° (median 610) for control lesion margins. Statistical analysis: p<0.0001, t-test. Scale bars: a, c, d, 100 μm; b, 50 μm.

FIG. 6 shows GDA transplantation suppresses neurocan and NG2 immunoreactivity. Panel (a) shows that at 4 days post injury, control lesion margins display dense neurocan immunoreactivity mainly associated with fine, GFAP− processes and to a lesser extent with GFAP+ astrocyte cell bodies. Panel (b) shows neurocan immunoreactivity at 4 days post injury/transplantation is greatly reduced in margins of hPAP+ GDA transplanted lesions. Panel (c) shows that at 8 days post injury/GDA transplantation, neurocan immunoreactivity within lesion margins has increased compared to the 4 day time point. Note however that intralesion hPAP+ GDAs continue not to be neurocan immunoreactive. Panels (d), (e) and (f) show GDA transplanted lesion centers (panels (e) and (f)) at 4 post injury display a marked reduction in NG2 immunoreactivity compared to control lesions (panel (d)). Panels (g), (h) and (i) show that although overall NG2 immunoreactivity has increased within the center of GDA transplanted lesions at 8 days post injury (panels (h) and (i)) compared to the 4 day time point (panels (e) and (f)), it is reduced compared to the more uniformly distributed NG2 immunoreactivity within the center of control lesions at 8 days post injury. Scale bars: a, b, g 100 μm; c, h, i, 50 μm;

FIG. 7 shows transplanted GDAs promote regeneration of rubrospinal axons. Panel (a) is a confocal montage scanned through a depth of 60 μm showing a small population of BDA labeled rubrospinal tract (RST) axons that have traversed a GDA bridged lesion of the dorsolateral funiculus and entered caudal white matter at 8 days post injury. The majority of RST axons however have sprouted to within 300 μm of the lesion center (LC) but failed to extend beyond the site of injury. Note the absence of BDA labeled axons within the dorsal most regions of the injury site. Panel (b) is a confocal montage showing the complete failure of axotomized BDA+ RST axons to cross control lesions and that the majority of axons have remained within rostral lesion margins at a distance of 500 to 800 μm from the lesion center (LC). Survival, 8 days. Panel (c) shows that at 5 weeks post injury/transplantation, a similarly small population of BDA+ RST axons to those observed at 8 days have traversed GDA bridged injury sites and extended within caudal white matter, however BDA+ axons have also sprouted into the dorsal regions of the lesion center and even extended beyond the pial surface (arrowhead and panel (d)). Note the lower levels of GFAP immunoreactivity in more ventral regions of the injury margins and center, coincident with the presence of BDA+ axons. Panel (e) shows two examples of RST axons displaying growth cones within white matter 2 mm caudal to a GDA treated injury; survival 5 weeks. Note the collateral branch (*). Panel (f) is a confocal image of a BDA+ terminal field-like axonal plexus within layer 5 spinal cord gray matter, immediately adjacent to the dorsolateral funiculus. Scale bars: a, b, c 200 μm; d, 100 μm; e, 5 μm; f, 10 μm.

FIG. 8 shows GDA transplantation suppresses atrophy of red nucleus neurons and promotes robust behavioral recovery. FIG. 8A is a graph showing that injured left hemisphere red nuclei in control rats contained an average of 52% of the neurons counted in uninjured right hemisphere red nuclei. Numbers of neurons in the injured left hemisphere red nuclei of GDA transplanted animals however had risen to 81% of neuron numbers in uninjured right hemisphere nuclei. Survival 5 weeks post injury (* p<0.01). FIG. 8B is a graph showing Gridwalk Analysis of Locomotor Recovery. Graph shows average number of mistakes per experimental group at different time points post injury.

FIG. 9 shows axon growth is aligned with processes of GDAs and host astrocytes in lesion margins Panel (a) (left) is a high magnification image of numerous GFP+ axons exiting the rostral host/graft interface of the dorsal column lesion shown in FIG. 3a. Panel (b) shows GFP-+axons are aligned with processes of host-GFAP+ astrocytes within the caudal lesion margin. Scale bars: a, 50 μm; b, c, 25 μm.

FIG. 10 shows GDA expression of astrocytic markers in vivo. At 4 days post injury/transplantation, the majority of hPAP+ GDAs within dorsal column lesion centers and margins display immunoreactivity for S100 (panels (a) and (b)) and vimentin (panels (d) and (e)). Sequential scanned confocal images through 2.5 μm depth (panels (c) and (f)) show co-localization of S100 panel (c)) and vimentin (panel (f)) with hPAP+ GDAs. Scale bars 25 μm.

FIG. 11 shows GRP transplantation does not reduce host astrogliosis. Confocal image of the caudal margin (LM) of a dorsal column lesion at 8 days post injury in an animal that received GRP transplants. hPAP+ GRPs fill the injury site and migrate into adjacent host white matter, however the host astrocytes display misaligned, hypertrophic GFAP+ processes similar to those observed in control, untreated lesions. Scale bar 50 μm.

FIG. 12 shows misaligned host astrocytic processes in control lesions. Panels (a) and (c) to (f) are confocal images of caudal lesion margins at 8 days post injury from 3 control animals showing dense meshworks of hypertrophic GFAP+ processes of host astrocytes, typical of forming glial scar tissue. Panel (b) is a high power image of boxed area in panel (a). GFAP+ processes were randomly selected within the lesion margin and best fit lines traced over them using Image Pro Plus software. Adjacent GFAP+ processes were then identically traced and the angle between the paired lines calculated using Image Pro software. Quantitative analysis of host GFAP+ astrocytic processes revealed an average angle of 59.4° (s.d.+/−22, median=61°) between adjacent paired processes. Scale bars (a, c-f) 100 μm.

FIG. 13 shows linearization of host astrocytes. Panels (a) and (c) to (f) are confocal images showing a striking alignment of host GFAP processes in caudal lesion margins at 8 days post injury in 3 animals that have received GDA transplants. Panel (b) is a high power image of boxed area in panel (a). Average angles of only 11.60 (s.d.+/−12.6, median=7°) were recorded between adjacent host GFAP+ processes within margins of GDA transplanted injury sites. Scale bars (a, c-f) 100 μm.

FIG. 14 shows GDAs suppress atrophy of axotomized red nucleus neurons. Panel (a) is a brightfield image showing cresyl violet stained red nucleus neurons in the uninjured right hemisphere in a control, lesion+cyclosporine rat at 5 weeks post injury. Panel (b) shows atrophy of neurons in the injured left hemisphere red nucleus affects 48% of cresyl violet stained neurons. Images in panels (a) and (b) were obtained from right and left nuclei within the same 25 μm coronal tissue section. Panel (c) shows GDA transplantation rescues 65% of the neurons in the injured left hemisphere red nucleus. Image in panel (c) was obtained from the same rostro-caudal region of the red nucleus as the images shown in (a) and (b) Scale bar 100 μm.

FIG. 15 is a graph showing Grid Walk locomotor performance. Rats that received transplants of type-2 GDAs showed no recovery of locomotor function compared to untreated control rats at all time points post injury. In contrast, rats that received type-1 GDA transplants performed significantly better than controls at all time points and their behavior improved significantly between 3 and 28 days post injury (Two Way Repeated Measures ANOVA, p<0.05).

### DETAILED DESCRIPTION

Provided herein are compositions and methods for the treatment of spinal cord injury or other traumatic or degenerative conditions of the CNS, promotion of axon regeneration, suppression of astrogliosis, re-alignment of host tissues, and the delay of axon growth inhibitory proteoglycan expression. Thus, provided is a method of treating a CNS lesion in a subject, comprising administering to the lesion a composition comprising glial restricted precursor derived astrocytes or GDAs. The method can be used for the treatment of spinal cord injury or other CNS lesions. The method can also be used in CNS lesions in which it is desirable to promote regeneration and/or re-alignment of host tissues, modulate the CNS scarring response, and rescue neurons from atrophy and death, or any combination thereof.

As used herein, the term GDAs refers to glial fibrillary acidic protein (GFAP)+/A2B5− cells, also referred to herein as type-1 GDAs, unless type-2 GDAs (GFAP+/A2B5+ cells) are specifically referenced.

The limited success to date of stem cell and neural progenitor cell transplantation is likely due to the inflammatory environment of adult CNS injuries, which direct undifferentiated neural stem cells or glial progenitors to a scar astrocyte like phenotype (Alonso, et al., Glia 49:318-38 2005; Frisen, et al., J. Cell Biol. 131:453-64 (1995). Scar astrocytes are poorly supportive of axon growth (Groves, et al., Dev. Biol. 159:87-104 1993; McKeon, et al., J. Neurosci. 11:3398-411 1991).

The methods and compositions described herein provide an alternative to allowing the lesion environment to direct differentiation of stem or progenitor cells while still retaining the benefit of starting with an undifferentiated cell Provided herein is a method of treating a CNS lesion in a subject, comprising administering to the lesion a composition comprising glial restricted precursor derived astrocytes (GDAs). The term lesion is used herein to refer to the site of injury to the CNS, the site of a CNS disease process, degenerative damage, or scarring, wherein promotion of regeneration would provide benefit. An important aspect of the provided method is that, prior to administration to the site of injury, GDAs are pre-differentiated from ancestral precursor cells, such as GRPs, using bone morphogenetic protein (BMP) family members, such as for example, BMP-4. GDAs can also be pre-differentiated from other ancestral precursor cells, including, but not limited to, restricted astrocyte progenitor cells or neuroepithelial stem cells. GRPs can be derived from pluripotential stem cells, such as mesenchymal stem cells and ES cells (Mujtaba, et al., Dev. Biol. 214:113-27 1999) and neuroepithelial cells (Rao and Mayer-Proschel Dev. Biol. 188:48-63 1997), using methods known in the art.

Glial restricted precursor (GRP) cells (Gregori, et al., J. Neurosci. 22:248-56 2002; Rao, et al., Dev. Biol. 188:48-63 1997; Rao, et al., PNAS 95:3996-4001 1998) are the earliest arising progenitor cell population restricted to the generation of glia. GRP cells are derived from multipotent neuroepithelial cells, distinct from the Embryonic-Neural Cell Adhesion (E-NCAM) positive neuronal-restricted precursors, and represent one of the earliest characterized glial precursor cells in the CNS. Tripotential GRP cells have been isolated based on the expression of the cell surface marker A2B5 from the developing spinal cord and from adult neural tissue (Johansson, et al., Cell 96:25-34 1999; Horner, et al., J. Neurosci. 20:2218-28 2000; Yamamoto, et al., Exp. Neurol. 172:115-27 2001).

Clonal analysis demonstrated that these cells are able to generate oligodendrocytes and two distinct type of astrocytes (type 1 and type 2) when exposed to appropriate signals in vitro. GRP cells are restricted to the glial lineage in vivo as they are unable to generate neuronal phenotypes in an in vivo neurogenic environment. GRP cells survive and migrate in the neonatal and adult brain. Transplanted GRP cells differentiate into myelin-forming oligodendrocytes in a myelin-deficient background and also generate immature oligodendrocytes in the normal neonatal brain

Any cell culture technology can be used for the preparation of GDAs. As an example, A2B5+GRPs can be isolated from dissociated cell suspensions of radial glia of embryos or subventricular zone cells of adults using standard methods known in the art, such as, for example, flow cytometry or immunopanning. GRP cells can also be isolated using growth procedures that favor selection of these cells. GRPs can also be derived from pluripotential stem cells, such as mesenchymal stem cells, ES cells and neuroepithelial cells, or other stem cells capable of generating CNS glia, using methods known in the art.

GRPs can be maintained in culture in a suitable medium. For example, GRPs can be maintained in culture with approximately 0.1-100 ng/ml bFGF and SATO supplements on a mixed laminin/fibronectin substrate. In order to differentiate GRPs to GDAs, the GRPs can be exposed to, for example, approximately 1-100 ng/ml of recombinant BMP-4 (for approximately 7 days in culture) to differentiate them into GDAs. Also disclosed is the use of other members of the BMP family, or other signaling molecules that induce differentiation along the astrocyte pathway within the antigenic range of type-1 astrocytes, so long as the cells further express the favorable properties expressed by GDAs, as disclosed herein.

GDAs for use in the methods described herein can be generated by the method comprising isolating subventricular zone cells from the subject, purifying A2B5 positive GRPs, and culturing said cells with a BMP. The GDAs can also be derived by the method comprising contacting pluripotential or multipotential cells with a molecule that induces differentiation, purifying A2B5 positive GRPs from the cells, and culturing said cells with a BMP.

To ensure GDA suspensions for transplantation do not contain undifferentiated GRPs or cells with the phenotype of type-2 astrocytes, contaminating cell types can be removed from the suspension by, for example, immuno-panning with the A2B5 antibody. A small volume of the resulting suspension can be plated onto glass coverslips and labeled with antibodies to A2B5 and GFAP to verify a uniform type-1 astrocyte phenotype. For transplantation, GFAP positive/A2B5 negative GDAs can be suspended in a suitable medium such as, for example, Hanks Balanced Salt Solution, at a density of 10³-10⁶ cells/μL.

Provided herein are isolated cell populations comprising at least about an 80%, 85%, 90%, 95%, 96%, 97%, 98%, 99%, or 100% pure population of GDAs or any percent between 80 to 100%. As used herein, the term isolated refers to a cell or population of cells which has been separated from its natural environment, e.g., removal from a donor animal, e.g., human. The isolated cell or population of cells can be in the form of a tissue sample, e.g., an intact sheet of cells, e.g., a monolayer of cells, or it can be in a cell suspension. The term isolated does not preclude the use of these cells in combinations or mixtures with other cells. The term population is intended to include two or more cells. Cells in a population can be obtained from the same or different source(s). Thus, for example, the isolated cell population can comprise at least 90% GDAs. Thus the isolated can comprise at least 95% GDAs or at least 99% GDAs. In certain embodiments, the isolated cell population does not comprise type-2 astrocytes or GRPs. Optionally, the isolated cell population does not comprise pluripotential or multipotential stem cells, such as ES cells or neuroepithelial stem cells. However, the isolated cell population of the herein provided method can comprise at most about 0.01%, 0.05%, 0.1%, 0.5%, 1%, 2%, 3%, 4%, 5%, 6%, 7%, 8%, 9%, or 10% type-2 GDAs, pluripotential stem cells, multipotential cells, undifferentiated glial precursors (e.g., GRPs), or any combination thereof. Thus, for example, the isolated cell population can comprise less than 10% type-2 GDAs. Thus the isolated cell population can comprise less than 5% type-2 GDAs. The purity of a cell population can be determined by, for example, detecting markers specific for various cell types in culture and determining by visual observation the percentage of cell types in the population and by other methods described herein or known in the art. Also provided herein are compositions comprising the isolated cell populations.

GRPs, from which GDAs are to be derived for use herein, are not restricted by origin. Thus, autologous, allogeneic or xenogeneic GRPs can be used. Autologous progenitor cells can be harvested from the subventricular zone of the patients themselves. Allogeneic cells can be harvested from aborted embryos or from organ donors. Xenogeneic cells can be harvested from a pig, monkey, or any other suitable mammal. Since the CNS is immunologically privileged site, transplanted xenogeneic cells can survive when a small amount of immunosuppressant drugs is administered. In addition to being derived from subventricular zone cells, GRPs can be derived from neuroepithelial stem cells, embryonic stem cells or other stem cells capable of generating CNS glia.

GRP-derived astrocytes (GDAs) generated by BMP exposure fall within the population of cells defined by their antigenic phenotype as type-1 astrocytes. In vitro studies on cells purified from the postnatal CNS have shown that type-1 astrocytes of postnatal origin promote extensive neurite growth from a variety of neurons in vitro (Baehr, Glia 3:293-300 1990; Noble, et al., J. Neurosci. 4:1892-1903 1984), express high levels of axon growth supportive molecules such as laminin/fibronectin (Gallo, et al., Exp. Cell Res. 187:211-23 1990) and NGF/NT-3-(Condorelli, et al., J. Mol. Neurosci. 6:237-48 1995) and also exhibit minimal chondroitin sulfate proteoglycan immunoreactivity in vitro. However, while transplantation of immature cortical astrocytes into adult brain injuries (Wunderlich, et al., Glia 10:49-58 1994) or acute adult spinal cord injuries (Wang, et al., Neurosci. 65:973-811995) have been shown to suppress astrogliosis, only limited sprouting of endogenous axons have been observed, with axons failing to penetrate the center of grafts or re-enter white matter beyond the sites of injury.

Thus, although GDAs show antigenic phenotypes like type-1 astrocytes, GDAs are a unique cell type that, when transplanted into CNS lesion sites, promote an unprecedented level of tissue reorganization, axon regeneration and locomotor recovery.

GDAs promote robust axon regeneration and functional recovery after transplantation into CNS lesion sites. The ability of GDAs to fill an injury site, suppress astrogliosis, re-align host tissues and delay expression of axon growth inhibitory proteoglycans indicate that these cells possess an effective ability to provide an axon regenerative environment. These attributes, in combination with their striking ability to significantly reduce atrophy of axotomized CNS neurons and support a robust behavioral recovery, make GDAs a highly effective cell type with which to repair a damaged or diseased CNS. Thus, the GDAs of the provided method can promote axon regeneration, suppress astrogliosis, re-align host tissues, delay expression of axon growth inhibitory proteoglycans, or any combination thereof.

No other cell population transplanted to date, including purified type-1 astrocytes, has the multiple and marked beneficial effects of GDAs. The level of axonal regeneration achieved with GDAs is otherwise only achieved with extensive additional manipulations. For example, rescue of the neurons of the red nucleus is only achievable with addition of brain-derived neurotrophic factor (BDNF). In this regard, this reveals another difference between GDAs and previously studied type-1 astrocytes, as previous studies indicate that astrocytes do not make BDNF, while readily detectable levels of BDNF mRNA in GDAs are disclosed herein. In addition, no other transplant, including that of GRP cells themselves, creates the beneficial tissue reorganization achieved with GDA transplantation.

The GDAs disclosed herein are administered using standard methods known in the art for use in the promotion of CNS nerve regeneration and/or scar reduction.

GDAs are used to treat subjects in which it is desired to promote CNS regeneration and/or reduce scar formation. Thus, GDAs can be applied in any conventional formulation to areas of a lesion.

There is no restriction to the location of a lesion. Thus, any part of the brain or spinal cord is treated. For example, the cerebral cortex, the mid-brain, the thalamus, the hypothalamus, the striatum, the substantia nigra, the pons, the cerebellum, the medulla, or any cervical, thoracic, lumbar, or sacral spinal segment. The method is applicable for any nervous system lesion including, for example, those caused by spinal cord injury (resulting, for example, in respiratory paralysis, quadriplegia, and paraplegia).

The GDAs can also be administered to patients in whom the nervous system has been damaged or injured by trauma, surgery, ischemia, infection, metabolic disease, nutritional deficiency, malignancy, toxic agents, paraneoplastic syndromes and degenerative disorders of the nervous system. Examples of such disorders include, but are not limited to, Alzheimer's Disease, Parkinson's Disease, Huntington's chorea, amyotrophic lateral sclerosis, progressive supranuclear palsy, and neuropathies. GDAs can be applied to a wound to reduce scar formation. Thus, after an operation, GDAs can be applied in order to reduce scar formation from lesions due to, for example, arterio-venous malformation, necrosis, bleeding, and craniotomy, which can secondarily give rise to epilepsy. GDAs can also be used for treatment of epilepsy, by stabilizing the epileptic focus and reducing scar formation.

Treatment can be performed, for example, within 24 hours, or alternatively, for example, one week, 5 years, or even more than 10 years after onset of the lesion. In cases where a lesion can be predicted, for example, surgery, the GDA can be delivered prior to or during the occurrence.

GDAs can be delivered by direct application, for example, by direct injection of a sample of GDAs into the site of nerve damage. For example, the spinal cord can be exposed by laminectomy, and a cellular suspension injected using a microsyringe under a surgical microscope. When high resolution MRI images are obtained, the cell suspension can be injected without laminectomy as in intervertebrally (e.g., by the technique of lumbar puncture).

Optimally, GDAs are delivered in a media which partially impedes their mobility so as to localize the GDAs to a site of lesion. By way of example, GDAs can be delivered in a paste or gel comprising, for example, a biodegradable gel-like polymer such as fibrin or a hydrogel. Such a semi-solid medium has the advantage that it impedes the migration of (scar-producing) undesirable mesenchymal components such as fibroblasts into the site.

Other methods of using and applying GDAs include, but are not limited to, the use of polymer implants and surgical bypass techniques. Such methods may be used together, alone, or in conjunction with other methods as described herein. Uses of polymer implants and surgical techniques are well known to those of skill in the art. For example, GDAs can be applied to a site of a lesion in a form in which the GDAs are seeded or coated onto a polymer implant. Various types of polymer implants can be used herein, with various compositions, pore sizes, and geometries. Such polymers include but are not limited to those made of nitrocellulose, polyanhydrides, and acrylic polymers (see e.g., those described in European Patent Publication No. 286284; Aebischer, et al., 1988, Brain Res. 454:179-187; Aebischar, et al., 1988, Prog. Brain Res. 78:599-603; Winn, et al., 1989, Exp. Neurol. 105:244-250, which are incorporated by reference at least for the polymers described therein).

Polymers can be used as synthetic bridges, over which nerve regeneration can be promoted and scar formation can be reduced by application of GDAs to the end(s), or in the vicinity of, the bridge. For example, an acrylic polymer tube with GDAs at one or more ends, or throughout the tube, can be used to bridge lesions rostrally or bypass lesions, e.g., of the spinal cord, over which regeneration can be induced. Semi-permeable tubes may be used, e.g., in the dorsal columns or dorsal afferents, which tubes can contain and provide for the release of trophic factors or anti-inflammatory agents. The types of tubes which can be used are well known to those of skill in the art.

Delivery systems for other compositions disclosed herein, such as neurotrophic factors, include administration by direct injections through catheters attached to indwelling osmotic pumps, through genetically engineered biological delivery systems such as transduced fibroblasts or immortalized cell lines, and by direct injection of genes or proteins into the spinal parenchyma at or near the lesion site.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

GDA can be immortalized by procedures known in the art, so as to preserve a continuing source of astrocytes or GDAs. Immortalized GDAs can be maintained in vitro indefinitely. Various methods of immortalization are known in the art including, but not limited to, viral transformation (e.g., with SV40, polyoma, RNA or DNA tumor viruses, Epstein Barr Virus, bovine papilloma virus, or a gene product thereof) and chemical mutagenesis. The cell line can be immortalized by a virus defective in replication, or is immortalized solely by expression of a transforming virus gene product. For example, GDAs can be transformed by recombinant expression vectors which provide for the expression of a replication-defective transforming virus or gene product thereof. Such procedures are known in the art.

GDAs can be cryopreserved. Various methods for cryopreservation of viable cells are known and can be used (see, e.g., Mazur, 1977, Cyrobiology 14:251-272; Livesey and Linner, 1987, Nature 327:255; Linner, et al., 1986, J. Histochem. Cytochem. 34(9):1123-1135; U.S. Pat. No. 4,199,022 to Senkan et al.; U.S. Pat. No. 3,753,357 to Schwartz; U.S. Pat. No. 4,559,298 to Fahy, which are incorporated by reference at least for the methods described therein).

Several agents have been applied to acute spinal cord injury (SCI) management and CNS lesions that can be used in combination with the herein provided compositions and methods. Such agents include agents that reduce edema and/or the inflammatory response. Exemplary agents include, but are not limited to, steroids, such as methylprednisolone; inhibitors of lipid peroxidation, such as tirilazad mesylate (lazaroid); and antioxidants, such as cyclosporin A, EPC-K1, melatonin and high-dose naloxone. Thus, the herein provided compositions can further comprise methylprednisolone, tirilazad mesylate, cyclosporin A, EPC-K1, melatonin, or high-dose naloxone or any combination thereof.

The compositions provided herein can also comprise, glutamate receptor antagonists including, but not limited to, the noncompetitive N-methyl-D-aspartate (NMDA) ion channel blocker MK-801 (dizocilpine, Merck & Co., Inc., Whitehouse Station, N.J.), 1,2,3,4-tetrahydro-6-nitro-2,3-dioxobenzo[f]quinoxaline-7-sulfonamide (NBQX), gacyclidine (GK-11, Beaufour-Ipsen, Paris, France), and agmatine.

Anti-inflammatory agents, such as, for example, CM101, cytokine IL-10, and selective cyclooxygenase (COX)-2 inhibitors can be used in conjunction with the methods and compositions described herein. Thus, the herein provided compositions can further comprise CM101, IL-10, or a selective COX-2 inhibitor or any combination thereof.

The compositions and methods described herein can also be used in conjunction with inhibitors of apoptosis, such as caspase inhibitors, for example, Bcl-2, and calpain inhibitors.

The compositions and methods described herein may comprise exogenous neurotrophins, including, but not limited to, nerve growth factor (NGF), glial-derived neurotrophic factor (GDNF), cilliary neurotrophic factor (CNTF), neurotrophic factor-3 and 4/5 (NT-3, NT-4/5), fibroblastic growth factor (FGF), and brain-derived neurotrophic factor (BDNF) or any combination thereof.

Inhibitors of netrins, semaphorins, ephrins, tenascins, integrins, and chondroitin sulfate proteoglycans (CSPG) can be used in the compositions herein. For example, chondroitinase can be used to remove CSPG. Thus, the herein provided compositions can further comprise an inhibitor of netrins, semaphorins, ephrins, tenascins, integrins, or CSPG. Thus, the herein provided compositions can further comprise a chondroitinase.

The compositions described herein may comprise, the IN-1 antibody, which neutralizes the inhibitory protein activity of NoGo, the myelin-derived growth-inhibitory protein, myelin-associated glycoprotein (MAG) or any combination thereof.

Approaches and agents that act through direct intracellular mechanisms in the nerve cell body to promote neurite growth can be used. Thus, inosine, a purine nucleoside, and cAMP and the compound AIT-082, a synthetic hypoxanthine derivative containing a para-aminobenzoic acid moiety (e.g., Neotrofin; NeoTherapeutics, Newport Beach, Calif.) can be used in the compositions and methods herein. Thus, the herein provided compositions can further comprise AIT-082.

Gene therapy allows the engineering of cells, which combines the therapeutic advantage of the cells in combination with a gene delivery system. For example, if delivery of neurotrophins is desired, cells that form myelin and secrete neurotrophins can be engineered to both promote neurite growth and restore nerve function.

Macrophages from the patient's own blood (autologous macrophages) can be activated and implanted at the site of the spinal injury. The patient's own activated macrophages can scavenge degenerating myelin debris, rich in non-permissive factors, and thus encourage regenerative growth without eliciting an immune response.

Axon fibers that demonstrate regenerative growth or collateral sprouting encounter an inhibitory environment as well as a physical gap that requires a permissive bridging substance. Thus synthetic bridges can be used in the methods described herein. Advances in the field of biomatrix material have provided opportunities to bridge the gap with artificial material, such as biodegradable hydrogels, or combinations of hydrogels and cells, that may promote regeneration. Desired properties of a synthetic bridge are to provide simultaneously a physical substrate for axonal attachment and growth without triggering antigenic host reactions.

The herein provided methods and compositions can further comprise immuno-suppressive drugs such as cyclosporins, tacrolimus (FK505), cyclophosamid, azathioprines, methotrexate, mizoribin alone or in any combination or the use thereof. Administration can be prior to or concurrent with or after the injection of GDAs. Thus, the herein provided compositions can further comprise cyclosporins, tacrolimus (FK505), cyclophosamid, azathioprines, methotrexate, or mizoribin.

The methods described herein can further comprise as a separate step, administration of an agent or compound prior to, during or after administration of the GDAs. The compositions and methods described herein may comprise the aforementioned agents in any combination. By way of example, the compositions containing GDAs described herein may also comprise a glutamate receptor antagonist and a neurotrophin. One or more of the aforementioned agents can be formulated with the GDA containing composition or may be administered separately from the GDA containing compositions described herein. If administered separately, the one or more additional agents can be administered before, after or simultaneously with the GDA containing compositions as appropriate.

Any combination of agents, compounds or therapies can be combined with the GDA compositions and methods described herein even if not explicitly mentioned as a combination. For example, combinations of immunosuppressive drugs and GDAs can further include any other agent mentioned herein (e.g., bridges, neurotrophic factors and/or anti-inflammatory agents).

The number of GDAs to be injected depends on the species, age, weight and the extent of the lesion(s). Usually it ranges from about 10³-10⁸, including 10³-10⁵, 10⁵-10⁸, 10⁴-10⁷, cells or any amount in between in total for an adult patient.

The substances provided herein can be delivered at effective amounts or concentrations. An effective concentration or amount of a substance is one that results in treatment or prevention of lesions of the CNS, promotion of axon regeneration, suppression of astrogliosis, re-alignment of host tissues, and the delay of axon growth inhibitory proteoglycans expression. The term therapeutically effective means that the amount of the composition used is of sufficient quantity to ameliorate one or more causes or symptoms of a disease or disorder. Such amelioration only requires a reduction or alteration, not necessarily elimination.

Effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. The exact amount of the compositions required will vary from subject to subject. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

The provided GDAs can be prepared for application by making cell suspensions of the cultured GDAs in a culture medium or a pharmaceutically acceptable carrier. Cell density for application can be from about 10³-10⁶ cells/μL. Thus, provided herein is a pharmaceutical composition comprising an effective amount of the disclosed GDAs in a pharmaceutically acceptable carrier. By pharmaceutically acceptable is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject, as would be well known to one of skill in the art. Such pharmaceutically acceptable carriers include sterile biocompatible pharmaceutical carriers, including, but not limited to, saline, buffered saline, dextrose, and water.

The agents may be incorporated into microparticles, liposomes, or cells. Any of the microparticles, liposomes or cells, including the GDAs, may be targeted to a particular cell type via antibodies, receptors, or receptor ligands. Targeting can be accomplished by various means known to those of skill in the art, including, for example, by way of genetic engineering.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution can be from about 5 to about 8 or from about 7 to about 7.5. Further carriers include sustained release preparations such as semipermeable matrices of solid hydrophobic polymers, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. Certain compositions can be administered, for example, orally, intramuscularly, subcutaneously, intravenously, intraventricularly, according to standard procedures used by those skilled in the art.

Pharmaceutical compositions may include carriers, thickeners, diluents, buffers, preservatives and surface active agents. Pharmaceutical compositions may also include one or more active ingredients such as antimicrobial agents, anti-inflammatory agents, and anesthetics.

Preparations for parenteral administration include sterile aqueous or non-aqueous solutions, suspensions, and emulsions. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers and electrolyte replenishers (such as those based on Ringer's dextrose). Preservatives and other additives may also be present such as for example, antimicrobials, anti-oxidants, chelating agents, and inert gases.

Disclosed herein are kits that are drawn to reagents that can be used in practicing the methods disclosed herein. The kits can include any reagent or combination of reagents that would be understood to be required or beneficial in the practice of the disclosed methods. For example, the kits could include GDAs or precursors thereof discussed herein, as well as the buffers and enzymes required to use them. Other examples of kits, include GDAs described herein, as well as neurotrophic factors, such as NGF, as well as the buffers and enzymes required to use them. Optimally kits include GDAs and instructions to use the same in the methods described herein.

The disclosed methods and compositions are applicable to numerous areas including, but not limited to, the treatment of CNS lesions. The disclosed compositions and methods can also be used in a variety of ways as research tools. Other uses are disclosed, apparent from the disclosure, and/or will be understood by those in the art.

As used in the specification and the appended claims, the singular forms a, an and the include plural referents unless the context clearly dictates otherwise. Thus, for example, reference to a pharmaceutical carrier includes mixtures of two or more such carriers.

Disclosed are materials, compositions, and components that can be used for, can be used in conjunction with, can be used in preparation for, or are products of the disclosed method and compositions. These and other materials are disclosed herein, and it is understood that when combinations, subsets, interactions and groups of these materials are disclosed that while specific reference of each various individual and collective combinations and permutation of these compounds may not be explicitly disclosed, each is specifically contemplated and described herein. For example, if a cell is disclosed and discussed and a number of modifications that can be made to a number of molecules including the cell are discussed, each and every combination and permutation of cell and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific element or combination of elements of the disclosed methods, and that each such combination is specifically contemplated and should be considered disclosed.

Ranges can be expressed herein as from about one particular value, and/or to about another particular value. When such a range is expressed, this includes a range from the one particular value and/or to the other particular value. It will be further understood that the endpoints of each of the ranges are significant both in relation to the other endpoint, and independently of the other endpoint. It is also understood that there are a number of values disclosed herein, and that each value is also herein disclosed as about that particular value in addition to the value itself. For example, if the value 10 is disclosed, then about 10 is also disclosed. It is also understood that each unit between two particular units are also disclosed. For example, if 10 and 15 are disclosed, then 11, 12, 13, and 14 are also disclosed.

As used throughout, by a subject is meant an individual. Thus, the subject can include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject can be a mammal such as a primate or a human.

As used herein treating or treatment does not have to mean a complete cure. It can also mean that one or more symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

When the terms prevent, preventing, and prevention are used herein in connection with a given treatment for a given condition (e.g., prevention of a CNS lesion), they mean that the treated subject either does not develop an observable level of the condition at all, or develops it more slowly and/or to a lesser degree than he/she would have absent the treatment. These terms are not limited solely to a situation in which the subject experiences no aspect of the condition whatsoever. For example, a treatment can be said to have prevented the condition if it is given during exposure of a subject to a stimulus that would have been expected to produce a given manifestation of the condition, and results in the subject's experiencing fewer and/or milder symptoms of the condition than otherwise expected. A treatment can prevent lesions of the CNS, for example, by resulting in the subject's displaying only mild overt symptoms of the lesion.

Throughout the description and claims of this specification, the word comprise and variations of the word, such as comprising and comprises, means including but not limited to, and is not intended to exclude, for example, other additives, components, integers or steps.

Optional or optionally means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed method and compositions belong. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### EXAMPLES

### Example 1

### GDA Transplantation Promotes Axon Regeneration

### Methods

### Isolation of GRPs and Generation of GDAs

A2B5+GRPs were isolated by Fluorescence Activated Cell Sorting (FACS) of dissociated cell suspensions from spinal cord of E13.5 transgenic Fischer 344 rat embryos expressing the gene for human placental alkaline phosphatase under the control of ROSA26 promoter (TgN(R26ALPP)14EPS) (Kisseberth, et al., Dev. Biol. 214:128-38 1999). GRPs were maintained in culture with DMEM-F12 media (Invitrogen, Inc., Carlsbad, Calif.) with 10 ng/ml bFGF (Sigma, St. Louis, Mo.) and SATO supplements on a mixed laminin/fibronectin substrate and exposed to 10 ng/ml of human recombinant BMP-4 (R&D Systems, Minneapolis, Minn.) for 7 days in culture to differentiate them into type-1 astrocytes.

### In Vitro Characterization of GDA Phenotype

Cells in culture were labeled live with anti-A2B5 or anti-NG2 antibodies (Chemicon, Temecula, Calif.), then fixed with cold acid/alcohol and labeled with anti-GFAP (Sigma, St. Louis, Mo.), anti-FGFR3 (Sigma, St. Louis, Mo.), or anti-p1p/DM20 (Chemicon, Temecula, Calif.) antibodies. Secondary antibodies were purchased from Jackson Immunologicals (West Grove, Pa.) and Molecular Probes (Eugene, Oreg.). BMP-induced GDAs were uniformly immunoreactive for human alkaline phosphatase in vitro. While no NG2+ or proteolipid protein/DM20+ oligodendrocyte precursors were detected in these cultures, occasionally undifferentiated GRPs (A2B5+/GFAP−) or type-2 astrocytes (A2B5+/GFAP+) were detected after BMP-4 treatment. These cell types represented less than 1% of the total cell population. To ensure GDA suspensions for transplantation did not contain undifferentiated GRPs or cells with the phenotype of type-2 astrocytes, potential contaminating cell types were removed from the suspension by immuno-panning with the A2B5 antibody. A small volume of the resulting suspension was plated onto glass coverslips and labeled with antibodies to A2B5 and GFAP to verify a uniform type-1 astrocyte phenotype. For transplantation, 100% GFAP positive/A2B5 negative GDAs were suspended in Hanks Balanced Salt Solution at a density of 30,000 cells/μl.

### Lesion Models and Cell Transplantation

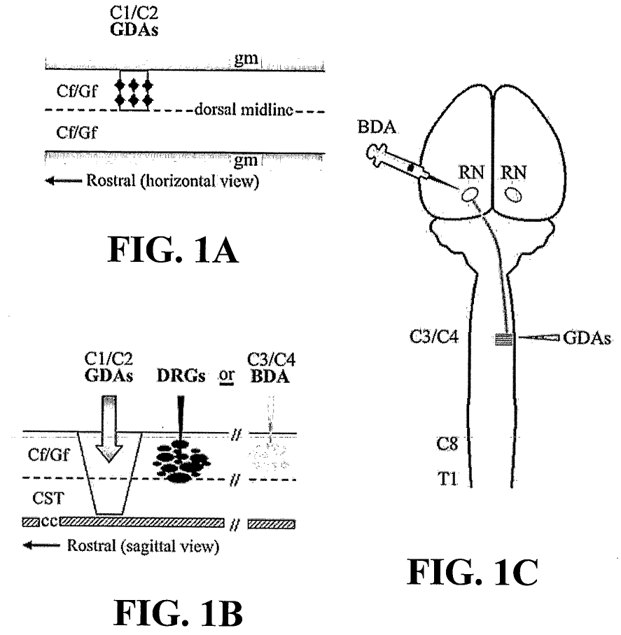

Adult female Sprague Dawley or Fischer 344 rats (3 months old, Harlan, Indianapolis, Ind.) were anesthetized by injection of a cocktail containing ketamine (42.8 mg/ml), xylazine (8.2 mg/ml), and acepromazine (0.7 mg/ml). For dorsal column injuries (FIGS. 1A and 1B), the right side dorsal column was unilaterally transected between cervical vertebrae 1 and 2 using a 30 gauge needle as a blade. Lesions extended to a depth of 1 mm and extended laterally 1 mm from the midline. For rubrospinal tract lesions, unilateral transections of the right side dorso-lateral funiculus including the rubrospinal pathway were conducted at the C3/C4 spinal cord level with Fine Science Tools micro-scissors. Lesions extended to a depth of 1 mm and extended medially 1 mm from the lateral pial surface of the spinal cord (FIG. 1C).

A total of 4 μl of GDA or GRP suspension (30,000 cells/μl; 120,000 cells) per animal was acutely transplanted into 6 different sites in dorsal column lesions i.e. two injections each into medial and lateral regions of the rostral and caudal lesion margins, and two injections into medial and lateral regions of the lesion center (FIG. 1A). All dorsal column in vivo experiments were conducted in the absence of immunosuppressants. GDA transplants were injected in an identical pattern into lesions of the dorsolateral funiculus and a total of 6 μl of GDA suspension (30,000 cells/μl; 180,000 cells) injected per injury site. Control lesion rats were injected with 6 μl of Hanks Balanced Salt Solution. As the experimental endpoint for many rats in the dorsolateral funiculus group was 5 weeks post injury/transplantation, all rats received daily injections of Cyclosporine (1 mg/100 g body weight) beginning the day before transplantation through the course of the experiment. Sham operated rats in which the spinal cord was exposed but not lesioned, and rats that received a lesion but no Cyclosporine were included as control groups.

Scar formation and CSPG expression after spinal cord injury were characterized in adult Sprague Dawley rats, a strain commonly used in CNS regeneration studies. Unilateral dorsal column stab injuries, reliably generated lesions of uniform size and induced consistent, quantifiable changes in CSPG expression in adult female Sprague Dawley rats. However, as the GDAs were derived from hPAP Fischer 344 rats, an initial pilot series was conducted of intralesion GDA transplants versus untreated controls in dorsal column injuries of both adult female Fischer 344 and Sprague Dawley rats and the ability of axons growing from adjacent dorsal root ganglion (DRG) neuron transplants to cross sites of injury was assayed versus controls that did not receive GDAs. Although GFP+ axons consistently failed to cross control lesions in both strains of rats, significant variations were observed in lesion size and margin morphology in control Fischer 344 rats, a phenomenon not observed in Sprague Dawley rats. The greater variation in lesion size and rostro-caudal distances of lesion margins from lesion centers in Fischer 344 rats precluded accurate quantification of the numbers of endogenous, BDA labeled axon growth at set distances from lesion centers in this strain of rats. Therefore, a separate study to investigate and quantify BDA+ endogenous sensory axon regeneration and CSPG expression in GDA transplanted versus control dorsal column lesion animals was conducted in Sprague Dawley rats. See Table 1 for rat strains and numbers of animals used in each study. Thus bridging dorsal column lesions with GDAs in two different strains of rat, in two separate axon regeneration experiments, both resulted in robust axon growth across sites of injury and failure of axons to traverse control injuries.

TABLE 1
Numbers of animals per experimental group.
Experiment 1: Analysis of endogenous sensory axon regeneration,
CSPG expression, and GDA phenotype

| Strain | Time point | Control | +GDA |
|---|---|---|---|
| Sprague Dawley | 4 days | 4 | 6 |
| | 8 days | 5 | 6 |

Experiment 2: Analysis of axon growth from GFP+ transplanted
sensory neurons.

| | | | +GDA/ |
|---|---|---|---|
| Strain | Time point | Control | GRP |
| Fischer 344 | 8 days | 6 | 9 |
| | (GDA) | | |
| Sprague Dawley | 8 days | 4 | 4 |
| | (GDA) | | |
| | 8 days | 4 | 6 |
| | (GRP) | | |

Experiment 3: Analysis of rubrospinal tract (RST) axon growth,
red nucleus, and behavioral recovery; Cyc, Cyclosporine.

| Strain | Time point | Control | +GDA |
|---|---|---|---|
| Sprague Dawley | 8 days | 6 + Cyc | 6 + Cyc |
| | 5 weeks | 9 + Cyc | 9 + Cyc |
| | 5 weeks | 7 | |
| | 5 weeks | 7 | |
| | | (sham) | |

### Adult DRG Neuron Transplantation

Single cell suspensions of adult mouse sensory neurons were prepared from 10-12 week old transgenic mice expressing the gene for enhanced green fluorescent protein (eGFP). No growth factors were added to the neuron suspension. 500 nl of the neuron suspension (˜1,500 neurons/μl) was acutely microtransplanted into dorsal column white matter approximately 500 μm caudal to the lesion (FIG. 1B).

### Histology

At 4 days, 8 days and 5 weeks post surgery, animals were deeply anesthetized and transcardially perfused with 0.1M phosphate buffered saline (PBS) followed by 4% paraformaldehyde in 0.1M PBS. For frozen sectioning, dissected spinal cords were cryoprotected in a 30% sucrose/PBS solution at 4° C. overnight. Tissue was embedded in OCT (Optimal Cutting Temperature) compound (Sakura Finetek USA, Inc., Torrance, Calif.) and quickly frozen. Serial, 25 μm thick frozen sections were cut in the sagittal plane and air dried onto gelatin coated glass slides. For Vibratome sectioning, dissected spinal cords were post-fixed in 4% paraformaldehyde overnight, then embedded in 5% gelatin/5% agar. 75 μm thick serial sagittal sections were collected and processed as free-floating sections. All tissue sections were washed in PBS, blocked with 4% normal goat serum in solution with 0.1% Triton/PBS for 30 minutes, then incubated with appropriate primary antibodies in the blocking solution overnight at 4° C. Secondary antibody incubations were for 1 hour at room temperature.

The following primary antibodies were used: monoclonal anti-GFAP (Sigma, St. Louis, Mo.) and polyclonal anti-GFAP (Sigma, St. Louis, Mo.); monoclonal anti-vimentin (Chemicon, Temecula, Calif.); polyclonal anti-S100 (Dako, Carpinteria, Calif.); 1F6 anti-neurocan (Developmental Studies Hybridoma Bank, Iowa City, Iowa); polyclonal anti-NG2 (Chemicon, Temecula, Calif.); polyclonal anti-GFP (Molecular Probes, Eugene, Oreg.); monoclonal anti-hPAP (Sigma, St. Louis, Mo.); polyclonal anti-hPAP (Fitzgerald, Concord, N.H.); CS56, chondroitin 6 and 4 sulphate proteoglycans (Sigma, St. Louis, Mo.). Cy5, Cy2 (Jackson Immunologicals, West Grove, Pa.), Alexa-488 and Alexa-594 (Molecular Probes, Eugene, Oreg.) conjugated secondary antibodies were used to visualize primary antibody binding. All secondary antibodies were pre-absorbed against rat serum. To control for non-specific secondary antibody binding, adjacent sections were also processed as described above without primary antibodies. Labeled sections were examined using an Olympus BX60 fluorescence light microscope and a Leica TCS SP2 confocal microscope. Molecule co-localization and cellular associations were determined with Leica 3D analysis software. All immunohistological images were acquired with confocal microscopy (Leica TCS SP2) of sections cut in the sagittal plane. Spinal cord rostral to the lesion is shown to the left in all figures.

### Tracing Endogenous Sensory or Rubrospinal Axons

In both lesion models, endogenous axons were traced by injection of 10% biotinylated dextran amine (BDA) in sterile PBS 8 days prior to an experimental endpoint. In the dorsal column lesion model, ascending endogenous axons were traced by BDA injection to a depth of 0.5 mm into the right-side, cuneate and gracile white matter at the C3/C4 spinal level (FIG. 1B). Descending rubrospinal tract axons were traced in the dorsolateral funiculus lesion model by injection of BDA into the magnocellular region of the left hemisphere red nucleus. For histological analysis of BDA labeled axons, 25 μm serial sagittal sections were collected and processed for immunohistochemistry as described above. BDA was visualized by incubating tissue sections with the VectastainABC solution (Vector Labs, Burlingame, Calif.), and further intensified with the Tyramide-Alexa 488 reagent (Molecular Probes, Eugene, Oreg.).

### Quantification of Endogenous Sensory Axons

The number of BDA labeled axons was counted in every third tissue section spanning the medial-lateral extent of dorsal column injury sites at the following locations: 0.5 mm caudal to the injury; directly at the injury center; 0.5 mm, 1.5 mm and 5 mm rostral to the injury site; and within the dorsal column nuclei. In order to control for differences in axon tracing/labeling efficiency between animals, the numbers of BDA labeled axons counted within the lesion center and at all rostral sites were normalized to the number of BDA labeled axons detected 0.5 mm caudal to the lesion site for each tissue section examined. The normalized values from each tissue section for each separate animal (control and GDA transplanted) were averaged to generate values for each animal. The values for each animal (n=6 GDA transplanted, 5 control) were then averaged and displayed graphically. ANOVA or t-tests were performed as appropriate, p<0.01. For separate experiments analyzing the growth of GFP+ axons from micro-transplanted sensory neurons, identical methods were used to count GFP+ axons from alternate sagittally orientated 75 μm vibratome sections.

### Quantification of Alignment of Host GFAP+ Astrocyte Processes.

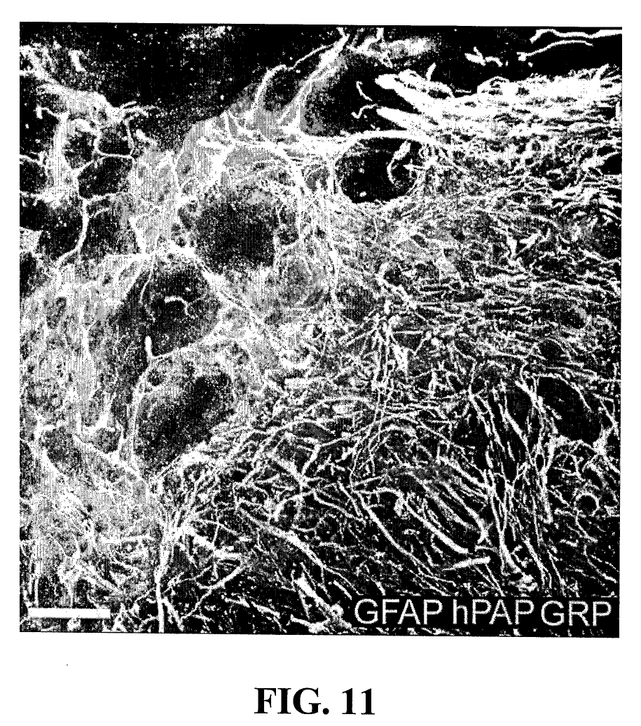

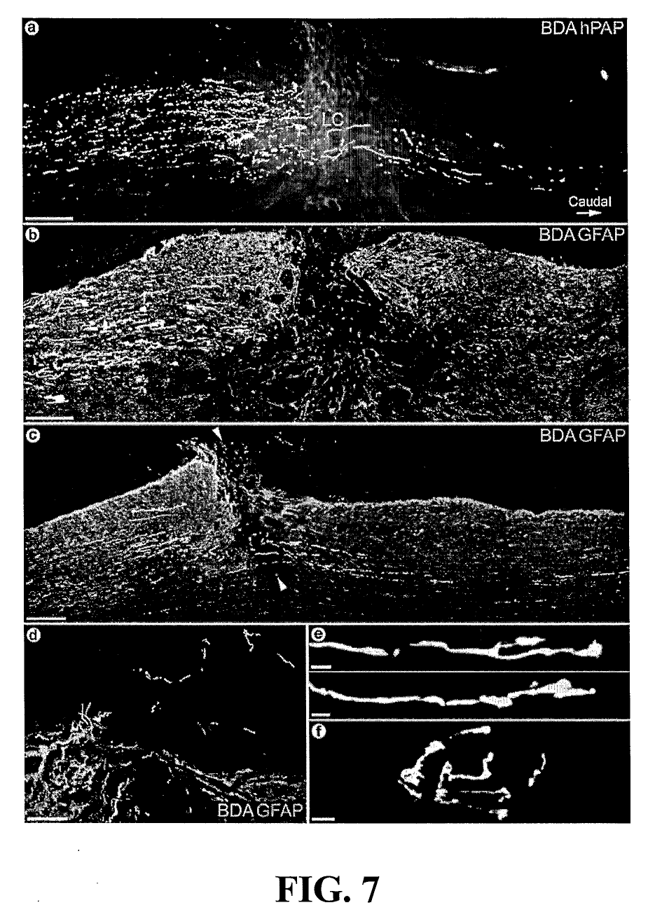

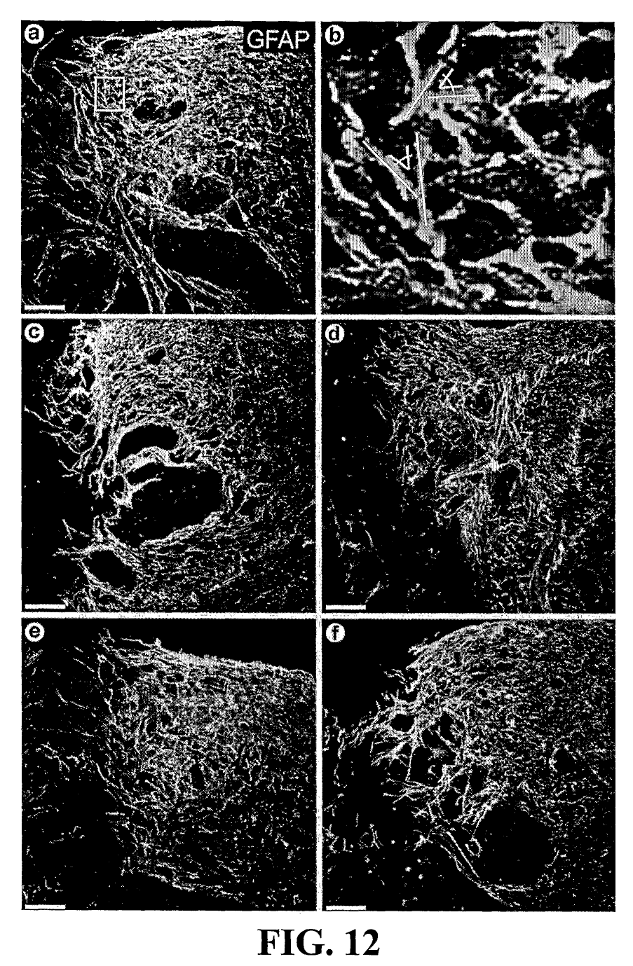

Confocal images were generated from scanning through 30 μm thick sagittal oriented sections of caudal and ventral dorsal column lesion margins immunostained for GFAP to show host astrocytic processes. Five sections were selected from the lateral to medial center of lesions in 3 control and 3 GDA transplanted rats (see FIGS. 10 and 11). Within each confocal image, GFAP+ processes were randomly selected within the lesion margin and “best fit” lines traced over them using Image Pro Plus software (Media Cybernetics, Silver Spring, Md.). Then an immediately adjacent GFAP+ process was identically traced and the angle between the lines calculated with the Image Pro software. In all, 20 pairs of GFAP positive host astrocytic processes from each confocal image (5 images per group) were analyzed and the mean and median angles were determined. A T-test was performed to determine the statistical significance of the difference in measured angles between astrocytic processes for GDA and control groups (p<0.0001).

### Grid Walk Behavioral Analysis

Two weeks prior to surgery, rats were trained to walk across a horizontal ladder (Foot Misplacement Apparatus, Columbus Instruments, Columbus, Ohio) and only rats that consistently crossed without stopping were selected for the study. The Grid Walk test is a sensitive measure of the ability of rats to step rhythmically and coordinate accurate placement of both fore and hind limbs. Trained rats were randomly assigned to one of four groups: rubrospinal tract (RST) lesion+GDA+ cyclosporine (n=9); RST lesion+suspension media+cyclosporine (n=9); RST lesion only (n=7); sham operation (n=7). One day prior to surgery (baseline) and at time points of 3, 7, 10, 14, 17, 21, 24, and 28 days post surgery, each rat was tested 3 times and the number of mis-steps from each trial was averaged to generate a daily score for each animal. Two Way Repeated Measures ANOVA and Tuley Post Test (p<0.05) were applied to analyze the data.

### Quantification of Red Nucleus Neurons

At 5 weeks post injury/transplantation, 25 μm serial frozen sections were cut in the coronal plane from the brains of rats that had undergone behavioral analysis. Every third section through the rostro-caudal extent of the red nucleus was stained with 0.2% cresyl violet. Standard, design-based stereology methods (CAST software, Olympus America Inc., Mellville, N.Y.) were used to quantify numbers of red nucleus neurons in each hemisphere of RST lesioned, GDA transplanted (n=6) and control (media injected/cyclosporine, n=6)) rats. An optical fractionator was applied to left and right hemisphere red nuclei from every 6^{th} section. Cell bodies greater than 2 μm in diameter and having characteristic neuronal morphology were counted. The numbers of neurons counted in the left hemisphere, injured red nucleus were normalized to counts obtained for the uninjured right hemisphere nucleus for each animal. The values for each animal within a group were averaged and displayed graphically. A T-test was performed to determine the statistical significance of the difference between the groups (p<0.01).

### Results

### Regeneration of Endogenous Sensory Axons

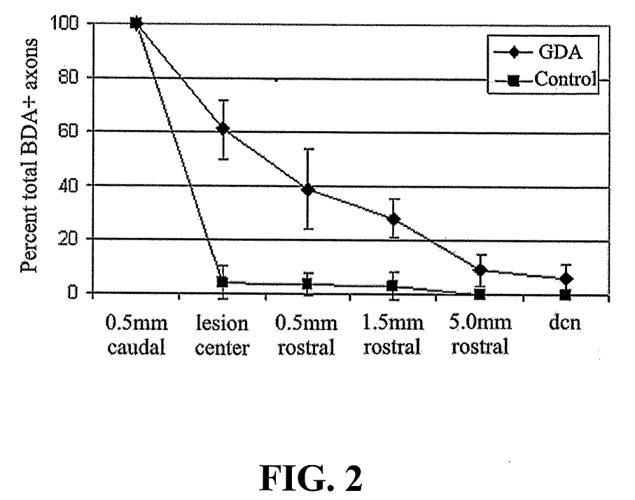

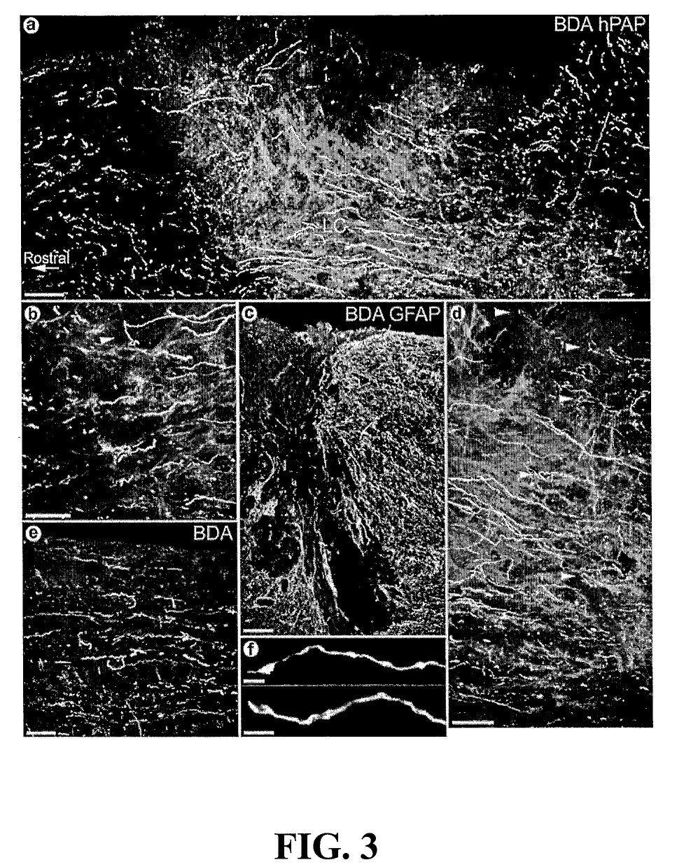

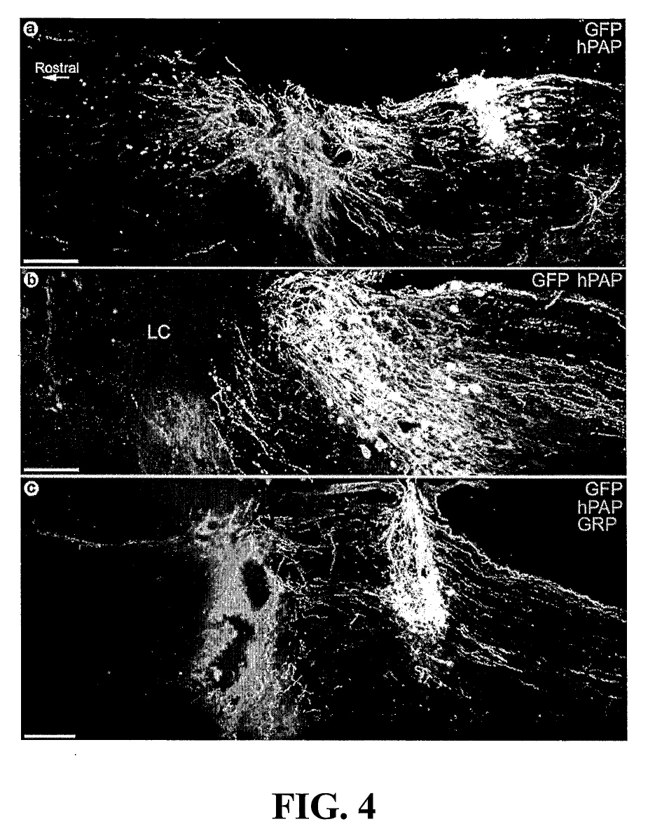

Transplantation of GRP derived astrocytes (GDAs) in stab wound lesions of the rat dorsal column white matter (FIG. 1A) resulted in the majority of axotomised sensory axons growing into the lesion center (FIG. 2 and panel (a) of FIG. 3), with 66% of these axons extending further beyond the lesion site into adjacent white matter (FIG. 2 and panels (a), (b), (d), (e) and (f) of FIG. 3). In order to minimize labeling of spared axons, a discreet population of ascending sensory axons aligned with the lesion site was traced en passage with a single biotinylated dextran amine (BDA) injection caudal to GDA-transplanted or control stab injuries of the right hand dorsal column cuneate and gracile white matter pathways (FIG. 1B). Sample counts from every third parasagittal section at 8 days post-injury, revealed similar numbers of BDA labeled axons 0.5 mm caudal to the injury site in both control and experimental spinal cords (107+/−47 vs. 101+/−45, respectively). In GDA transplanted cords, on average 61% (s.d+/−11) of caudal labeled axons extended into the lesion center, 39% (s.d.+/−15) of caudal axons extended 0.5 nm beyond the lesion center into adjacent white matter, 28% (s.d.+/−7) extended 1.5 mm beyond the lesion site. Even at the relatively short 8 day time point, small numbers of axons extended still further, with averages of 7 BDA+ axons (s.d.+/−5; i.e. 7%) detected per animal at 5 mm rostral to the injury site and 4 axons (s.d.+/−3; i.e. 4%) in the dorsal column nuclei in GDA transplanted animals. In contrast, in ⅘ control animals, no axons were observed within the lesion centers or within white matter beyond the lesion. In just ⅕ control animals, 6 BDA+ axons (4%) were found in the ventral most regions of the lesion site (i.e. at the ventral margin), effectively rostral to the caudal lesion margin and therefore aligned with the lesion center. These were most likely due to a limited axonal sparing/sprouting in this animal, resulting in the presence of these axons in the ventral white matter of the cuneate pathway at the interface with gray matter. That no BDA+ axons were observed beyond 1.5 mm rostral to the lesion in this animal (FIG. 2) or observed crossing the injury site near the pial surface or within GFAP negative regions of the lesion center proper in all control animals, supports that these 6 axons had sprouted around the injury at the gray/white matter interface rather than been spared. Overall, ˜99% of the cut ends of BDA+ axons in control cords had remained within caudal lesion margins and displayed dystrophic endings (FIG. 3 panel (c)). In sharp contrast, very few dystrophic axons were observed at the caudal interface of GDA transplants with adjacent white matter compared to control injury sites (compare panels (c) and (d) of FIG. 3).

### Axon Growth Supportive Gda Bridge

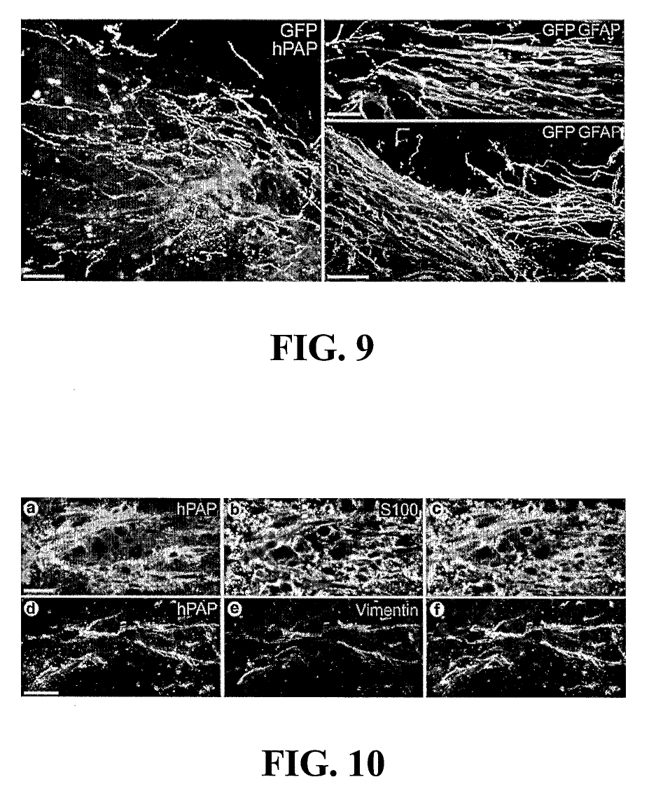

Although the presence of endogenous sensory axons at all points within lesion centers and rostral lesion margins in GDA transplanted cords indicated true regeneration rather than sparing, the ability of GDAs to support adult sensory axon growth across identical stab injuries was examined in an adult DRG neuron/GDA transplant model. In these experiments, a separate series of rats received microtransplants of adult mouse GFP+ sensory neurons that were injected acutely into dorsal column white matter 400-500 μm caudal to either GDA-transplanted (FIG. 1B and panel (a) of FIG. 4), GRP transplanted (FIG. 1B and panel (a) of FIG. 4a) or control media injected stab injuries. Newly growing axons from transplanted adult sensory neurons consistently fail to cross experimental dorsal column stab lesions. In contrast to the complete failure of axons to cross lesion sites in media injected or GRP transplanted animals at 8 days post injury/transplantation (panel (c) of FIG. 4), 53% (s.d.+/−3) of rostrally directed GFP+ axons had grown into lesion centers, 62% of axons at the lesion center reached 0.5 mm beyond GDA-filled lesion sites, 42% reached 1.5 mm into rostral white matter, with small numbers of axons extending up to 2 mm beyond the injury site (panel (a) of FIG. 4 and panel (a) of FIG. 9). Comparison of data from these separate experiments revealed a remarkably similar efficiency of axon growth (66% BDA+ axons, 62% GFP+ axons) exiting GDA filled injuries.

The importance of integration of GDAs into the lesioned spinal cord was indicated by the striking correlation between the extent of axonal growth with the degree of occupancy of the lesion by GDAs. In 2 GDA transplanted animals in which GDAs did not completely fill the lesion site, very few GFP+ axons penetrated the GDA-poor caudal lesion margins and GFP+ axons within lesion centers were confined to areas containing GDAs (panel (b) of FIG. 4). In areas of the lesions that were devoid of hPAP+ GDAs, GFP+ axons formed dystrophic endings within caudal lesion margins (panel (b) of FIG. 4). In these cases, no axons were observed to cross the site of injury and enter rostral white matter. In both sets of axon growth experiments, axons rarely fasciculated in white matter or within GDA-transplanted lesion centers. GFP+ axon growth was often aligned with hPAP+ processes of GDAs (panel (a) of FIG. 9), and parallel with the host GFAP+ astrocyte processes in the rostral and caudal lesion margins (panel (b) of FIG. 9). Similarly, BDA+ endogenous axons were invariably aligned with hPAP+ GDAs within rostral (panel (b) of FIG. 3) and caudal (panel (d) of FIG. 3) lesion margins.

### Linearization of Host Tissue

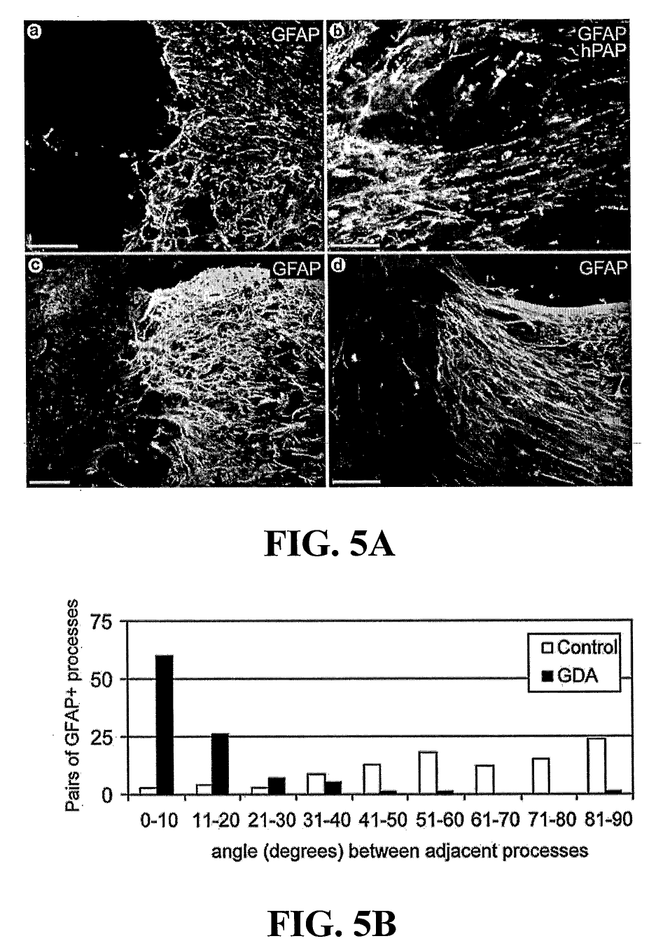

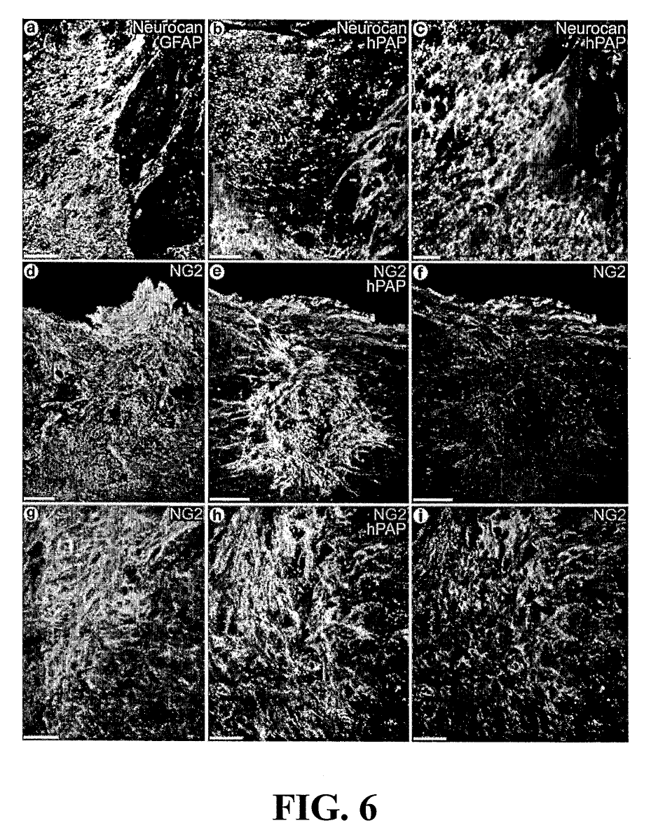

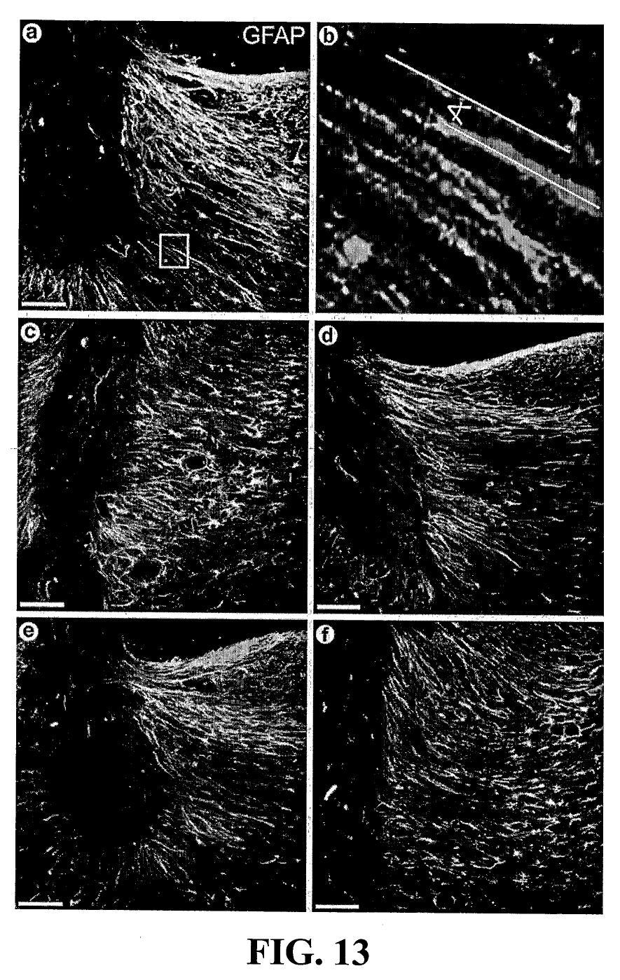

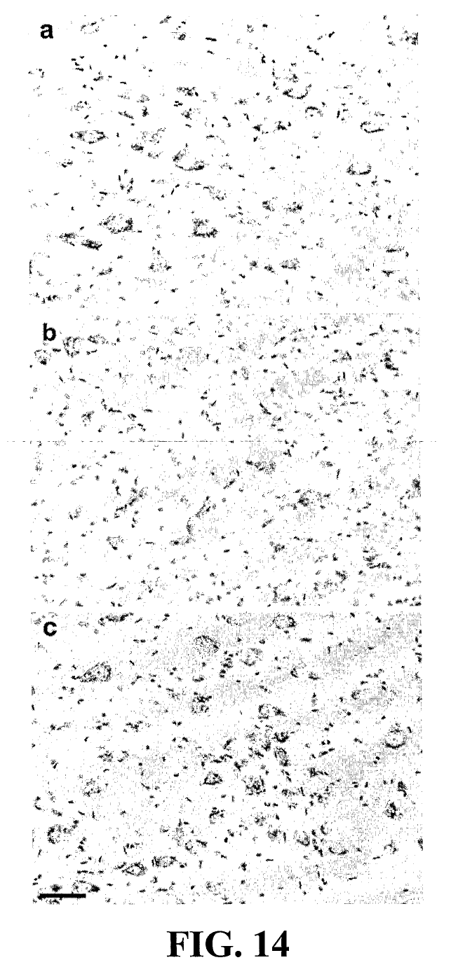

In both sensory axon regeneration experiments, a remarkable linearity of axonal growth was observed, particularly within lesion margins (panels (a) and (d) of FIG. 3 and panels (a) to (c) of FIG. 9) prompting examination of the underlying tissue organization. GDA transplantation was associated with a major reduction in astrogliosis, accompanied by a striking reorganization of host astrocyte cell bodies and processes within lesion margins compared to untreated controls (panels (a) to (d) of FIG. 5A). As GDAs were derived from animals expressing human placental alkaline phosphatase (HPAP), these cells were examined in conjunction with double-labeling for various cell-type specific antigens. To examine the host astrocytes, the down-regulation of GFAP in the transplanted GDAs (panel (b) of FIG. 5A) was used to identify host astrocytes with anti-GFAP immunostaining. Intra-lesion GDAs however remained S100+ and vimentin+, (panels (a) to (f) of FIG. 10) and did not express the oligodendrocyte lineage antigens NG2 (panels (e) and (h) of FIG. 6) or proteolipid protein. GFAP+ host astrocytes within the margins of control media injected or GRP transplanted spinal cord lesions exhibited the characteristic hypertrophic cell bodies of adult reactive astrocytes and had formed a dense mass of numerous, ramified, misaligned processes typical of astrogliotic scar tissue (panel (c) of FIG. 3, panels (a) and (c) of FIG. 5A and panel (a) of FIG. 11). In contrast, in animals receiving GDA transplants, host GFAP+ astrocyte processes within lesion margins were now oriented toward lesion centers (panels (b) and (c) of FIG. 5A and FIG. 13). Quantitative analysis of host GFAP+ astrocytic processes in control lesion margins revealed an average angle of 59.4° (s.d.+/−22, median=61) between adjacent pairs of astrocytic processes. However GDA filled lesions had average angles of only 11.6° (s.d.+/−12.6, median=7°) between adjacent host GFAP+ processes within lesion margins (FIG. 5B). GDAs within lesion margins often interweaved with endogenous GFAP+ astrocytes (FIG. 5b), creating an aligned and potentially more axon growth permissive transition between GDA bridged lesions and adjacent white matter (panel (d) of FIG. 5A and FIG. 5B) The ability of GDAs to re-align injured host spinal cord white matter is likely to have played an important role in facilitating the high efficiency of axon growth across GDA bridged injuries.

### Suppression of Inhibitory Proteoglycans

The generation of an axon growth permissive astrocytic terrain that did not physically resemble normal spinal cord scar tissue in rats that received GDAs was also associated with a delayed expression of axon growth inhibitory proteoglycans in the dorsal column lesion. The margins of control DC lesions examined 4 days post injury displayed a high density of neurocan immunoreactivity associated with numerous, fine, GFAP negative processes (panel (a) of FIG. 6), which were previously shown to be primarily associated with NG2+ glia (Tang, et al., J. Neurosci. Res. 71:427-44 2003). In addition, intense NG2 immunoreactivity in control lesions was predominantly associated with invading meningeal fibroblasts and blood vessels in the center of control lesions (panels (d) and (g) of FIG. 6). In contrast, margins of lesions containing GDA grafts at 4 days post injury displayed a marked reduction in overall neurocan immunoreactivity (panel (b) of FIG. 6) compared to controls (panel (a) of FIG. 6). This pattern of neurocan expression was similar to that previously observed at 2 days post injury in control lesions (Tang, et al., 2003). GDA transplanted injury sites also displayed greatly reduced NG2 immunoreactivity compared to controls at 4 days post injury (panels (e) and (f) of FIG. 6). At the 8 day time point, however neurocan immunoreactivity in the margins of GDA-transplanted lesions was similar in intensity and distribution to neurocan detected in control lesions at 8 days post injury (panel (c) of FIG. 6), indicating that the effect of the GDA transplant was to delay the expression of neurocan in lesion margins. Significantly however, GDAs within lesion margins and centers displayed little or no neurocan immunoreactivity (panel (c) of FIG. 6). Unlike the more uniform density of NG2 immunoreactivity within lesion centers at 8 days in control cords, NG2 immunostaining within GDA transplanted injuries had a more patchy distribution (panels (h) and (i) of FIG. 6). This reflects a chimeric mix of NG2 negative GDAs with host NG2 positive tissue at lesion centers (compare panels (g), (h) and (i) of FIG. 6), as hPAP+/NG2+ cells were not detected in the lesions even 8 days post transplantation. Analysis of CSPG deposition within GRP transplanted dorsal column injuries at 8 days post injury however revealed intense immunoreactivity both within lesion margins and centers for CS56 (a—general marker for 6 and 4 sulfated CSPGs) as well as neurocan and NG2.

### Rubrospinal Axon Regeneration

At 8 days post injury/GDA transplantation, 4 out 6 rats displayed GDA transplants that completely filled the sites of injury. Flares of migrating HPAP positive GDAs extended into lesion margins (panel (a) of FIG. 7), integrating and aligning GFAP+ host astrocyte processes in a similar fashion to that observed for successful GDA grafts within dorsal column injuries. In contrast to the complete absence of RST axons within the center of control lesions (panel (b) of FIG. 7), a significant population of BDA labeled RST axons were present within GDA transplanted lesion centers (panel (a) of FIG. 7) and within caudal white matter up to 1.5 mm beyond the site of injury. However the majority of axotomized RST axons within these animals were observed interacting with GDAs in rostral lesion margins and had only sprouted to within 300 μm of lesion centers (panel (a) of FIG. 7). The majority of BDA+ axons in control injury sites, displayed dystrophic endings and were between 500 and 800 μm from lesion centers (panel (b) of FIG. 7). In GDA bridged injuries, those axons that had grown into caudal white matter were invariably observed in the ventral half of the injury sites, which correlated with regions of GDA transplants that more often continuously spanned the injury site (panel (a) of FIG. 7). In the remaining 2 out of 6 animals in which GDA grafts did not span sites of injury (see also panel (b) of FIG. 4), no BDA axons were observed within white matter beyond the site of injury. In all six GDA transplanted rats and six media alone injected controls, BDA labeling was not observed within adjacent spinal cord gray matter beyond the injury site at 8 days post injury.

At 5 weeks post injury, hPAP positive GDAs were no longer detectable, however BDA+ RST axons were still observed within lesion centers and had extended further within caudal white matter up to 3 mm beyond sites of injury. Notably in 6 of 9 rats, RST axons were also now observed to have sprouted up within the more dorsal regions of lesion centers and to have even grown out into dorsal roots (panels (c) and (d) of FIG. 7). Growth cones were observed on axons within white matter caudal to the injury site (panel (e) of FIG. 7) clearly demonstrating that successful GDA transplants had stimulated RST axon regeneration beyond sites of injury. In 2 out of 9 rats, a few widely dispersed axon arborizations, similar in morphology to that seen for axons innervating terminal fields, were also now detected in gray matter at distances of 1 to 2 mm beyond the injury site (panel (f) of FIG. 7). Although still retaining a more rostro-caudal alignment than controls, host GFAP positive astrocytic processes were notably more hypertrophic within more dorsal lesion margins than ventral margins of GDA transplanted injuries (panel (c) of FIG. 7). In control rats that received lesions and cyclosporine but no GDA transplants, RST axons remained in the rostral lesion margins at 5 weeks post injury and displayed dystrophic endings.

### GDA Transplantation Suppresses Atrophy of Red Nucleus Neurons

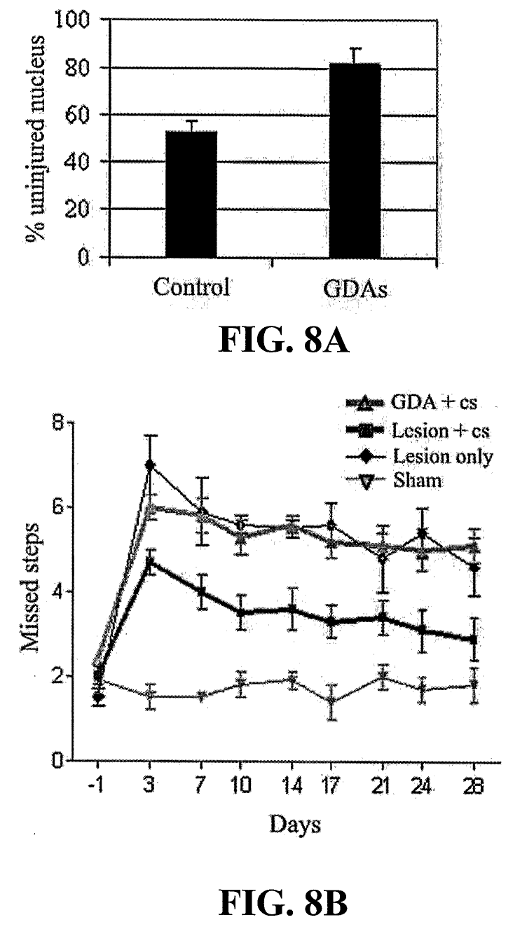

Neurons in the red nucleus begin to atrophy 1 week post injury after RST transection. Designed based stereological analysis revealed a striking 29% increase in the number of cresyl violet labeled neurons of greater than 20 μm cell body diameter detected in the injured red nuclei of GDA transplanted rats at 5 weeks post injury/transplantation compared to control rats. In GDA transplanted rats, the number of neurons in the injured left hemisphere red nucleus was reduced to an average of 81% of neuron numbers in the uninjured right hemisphere nucleus, compared to 52% in control rats (panel (a) of FIG. 8). GDA transplantation promotes behavioral recovery Transection of the dorso-lateral funiculus severs descending, supraspinal axons and results in chronic deficits in both fore- and hind-limb gross and fine motor function which can be detected by the Grid Walk behavioral test, Following transection of the dorso-lateral funiculus, rats that received GDA transplants performed significantly better than controls at all post surgery time points (panel (b) of FIG. 8) and their behavior improved significantly between 3 days and 28 days post injury (Two Way Repeated Measures ANOVA, p<0.05). Rats that received GDA transplants made an average 4.7 mistakes at 3 days post injury/transplantation and improved to an average of 2.9 mistakes at 28 days post injury. In contrast, control lesioned rats made on average 6 mistakes at 3 days post injury, and showed no statistically significant improvement at any later time point with an average of 5.1 mistakes at 28 days post injury. Prior to surgery, rats in control and treated groups performed equally well with a baseline average of 2.0 mistakes. Thus the average number of mistakes made by GDA transplanted animals had improved to a remarkable 0.9 points above baseline, representing a 70% recovery compared to control lesioned rats at 28 days post injury. Notably analysis of individual rats at 28 days showed that 4 out of 9 lesioned animals that received GDA transplants had scores that were statistically identical to their pre-surgery baseline scores.

### Example 2

### The Effects of Type-1 and Type-2 GDAs on Spinal Cord Glial Scar Formation

As described in Example 1, the transplantation of GDAs that were generated from GRP cells using BMP-4 as an inducing signal showed neurite outgrowth. Behavioral recovery, alignment of endogenous astrocytes and sparing of the red nuclei was seen in the lesion animals.

GRP cells generated GFAP+/A2B5− astrocytes (referred to as type-1 GDAs) in response to BMP but also generated an antigenically distinct astrocytes type that is GFAP+/A2B5+ (referred to as type-2 GDAs) in response to CNTF or LIF treatment in vitro. Therefore, the effects of intra-lesion transplants of type-1 or type-2 GDAs on spinal cord glial scar formation were compared. Particularly, the effects of transplant mediated changes in levels of axon growth inhibitory CSPGs and transforming growth factor betas at sites of injury were compared.

Confocal analysis of dorsal column stab injuries that had received acute transplants of type-1 GDAs revealed a marked suppression of immunoreactivity for the inhibitory CSPGs, neurocan and NG2 at 4 days post injury compared to media injected controls. However, by 8 days post injury, immunoreactivity for neurocan within lesion margins had increased in intensity to that observed for control injuries. Similarly, immunoreactivity for NG2 within lesion centers and margins of injuries receiving GDA type-1 had also increased significantly compared to that observed at 4 days post injury, although in this case the intensity of NG2 immunoreactivity was still reduced compared to media treated controls. Transplanted type-1 GDAs did not display neurocan immunoreactivity and had therefore retained a type-1 astrocytic phenotype. Transplanted type-1 GDAs down regulated their expression of GFAP, however their continued immunoreactivity for the astrocyte lineage markers S100 and vimentin confirmed retention of an astrocytic phenotype. Thus transplantation of type-1 GDAs to acute injuries promoted a significant but transient suppression of inhibitory CSPGs, effectively promoting axon growth across sites of injury.

In contrast to type-1 GDAs, transplanted type-2 GDAs did not suppress spinal cord scar formation. Confocal analysis of neurocan immunoreactivity at 8 days post injury for type-2 GDA bridged injuries showed that unlike type-1 GDAs, transplanted type-2 GDAs displayed significant numbers of neurocan+/hPAP+ cells, particularly in regions of the transplants adjacent to lesion margins. Transplanted type-2 GDAs also retained their phenotype with respect to CSPG expression. Similarly widespread neurocan expression associated with intra-lesion transplants of GRPs was observed at 8 days post injury. Neurocan is a potently inhibitory CSPG known to be expressed by endogenous reactive astrocytes within CNS injuries (Jones, et al., Exp. Neurol. 182:399-411 2003). Therefore neurocan expression is associated with GRP transplants containing GFAP+ cells and the environment of CNS injuries promotes the differentiation of glial precursors to an astrocytic phenotype that is poorly supportive of axon growth. The contrasting neurocan expression data for type-1 GDAs versus GRPs and type-2 GDAs corresponds with the respective ability of these different cell types to support axon growth and functional recovery after transplantation to acute spinal cord injuries.

In addition to the expression of axon growth inhibitors, another major feature of CNS scar tissue thought to contribute to the failure of axon regeneration is disruption of the linear cytoarchitecture of white matter pathways and the formation of a physical barrier presented by fibrotic glial scar tissue. A prominent indicator of glial scar formation is the meshwork of misaligned, hypertrophic astrocytic processes that forms within injury margins. Migration of type-1 GDAs out of the site of injury was associated with an alignment of host GFAP+ astrocytic processes within lesion margins. In order to quantify this effect a method was developed for measuring the angle between adjacent host GFAP+ processes within lesion margins which in the case of type-1 GDA transplanted injuries, took advantage of the down regulation for GFAP immunoreactivity by these cells. Analysis of the astrocytic process within the margins of cervical, dorsal column transection injuries revealed an average angle of 59.4° for control media injected injuries versus just 11.6° for host astrocytic processes in type-1 GDA treated injuries. In contrast, the margins of injuries transplanted with GRPs or type-2 GDAs displayed misalign astrocytic processes similar to those observed in control untreated injuries. Although alignment of astrocytic processes within CNS injuries has been shown to be insufficient to promote axon regeneration (Davies, et al., Exp. Neurol. 142:203-16 1996), the efficiency of axon growth across an injury site with reduced inhibitor expression was observed for injuries receiving type-1 GDAs.

### Example 3

### Axon Growth Across GDA Versus GRP Bridged Spinal Injuries

Transplantation of type-1 GDAs into stab injuries of the adult rat dorsal column pathways resulted in robust regeneration of about 40% of traced endogenous sensory axons across the injury site in just 8 days. In rats transplanted type-1 GDAs supported a similarly efficient growth of GFP labeled axons across the site of injury from adjacent transplants of adult DRG neurons. In contrast, intra-lesion transplants of undifferentiated GRPs resulted in the complete failure of GFP axons to traverse sites of injury. This demonstrates that pre-differentiation of glial precursors to an astrocytic phenotype is required for them to support axon growth across acute spinal cord injuries. Transplantation of type-2 GDAs to identical dorsal column transection injuries in adult rats also failed to provide an axon growth supportive bridge for newly growing GFP labeled sensory axons across acute injuries. Thus not all types of astrocytes that can be derived from embryonic glial precursors are equally supportive of axon growth in vivo.

To test the ability of type-1 GDAs to promote functional recovery, rats that received type-1 GDA transplants after transection of the dorsolateral funiculus performed significantly better than untreated controls (with or without cyclosporine) at all post surgery time points in the Grid Walk behavioral test and their behavior continued to improve significantly between day 3 and day 28 post injury (Two Way Repeated Measures ANOVA, p<0.05). Rats that received type-1 GDA transplants made an average 4.7 mistakes at 3 days post injury/transplantation and improved to an average of 2.9 mistakes at 28 days post injury. In contrast, control lesioned rats made an average of 6 mistakes at 3 days post injury, and showed no statistically significant improvement at any later time point with an average of 5.1 mistakes at 28 days post injury. Prior to surgery, rats in control and treated groups performed equally well with a baseline average of 2.0 mistakes. Thus the average number of mistakes made by type-1 GDA transplanted animals had improved to just 0.9 points above baseline, compared to no recovery of control lesioned rats at 28 days post injury. Moreover, analysis of individual rats at 28 days showed that 4 out of 9 lesioned animals that received type-1 GDA transplants had scores that were now statistically identical to their pre-surgery baseline scores. Thus type-1 GDA transplantation was associated both with an early neuroprotective effect at 3 days post injury, with further recovery in the 4 weeks following injury suggesting a possible contribution of RST axon regeneration and/or plasticity of spared supraspinal pathways e.g. CST and propriospinal circuits.

These data demonstrated the failure of transplanted GRPs and type-2 GDAs to suppress scar formation and support axon growth across acute spinal cord injuries. A study has shown the ability of GRPs to suppress glutamate mediated neurotoxicity in vitro (Maragakis, et al., Glia 50:145-59 2005). Therefore, there remained the potential for acutely transplanted GRPs to promote functional recovery via this mechanism, an effect that would be detectable during the first week post injury. An analysis of Grid Walk performance was conducted at time points ranging from 3 days to −2 weeks post injury in a further series of matched RST lesioned rats that received transplants of GRP cells, type-1 GDAs or control media injections. Type-1 GDA transplanted rats again showed a significant recovery of locomotor function compared to controls at all time points post injury (Two Way Repeated Measures ANOVA, p<0.05) with an average score of 1.5 (±0.2) mistakes compared to 5.9 (±0.2) mistakes for lesion only controls by day 14. In contrast, GRP transplanted animals showed no recovery of locomotor function compared to controls at all time points post injury. These results demonstrate that pre-differentiation of glial restricted precursors is required prior to transplantation to acute spinal cord injuries to promote functional recovery.

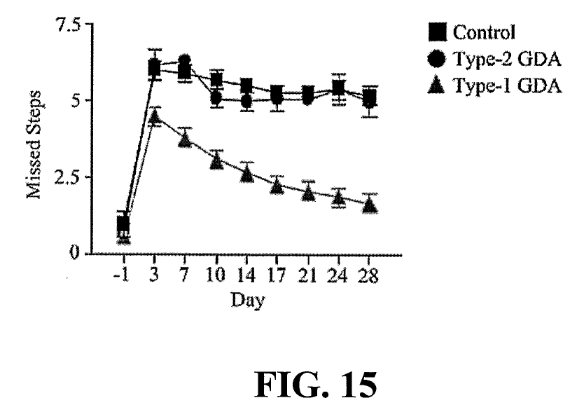

The ability of transplanted type-1 and type-2 GDAs to promote locomotor recovery was also compared in lesioned adult rats. Analysis of Grid Walk performance at time points ranging from 3 days to 4 weeks post injury, showed that type-1 GDA treated rats displayed a robust recovery of function with an extent and time course that closely matched that observed in previous experiments (FIG. 15). Rats receiving acute transplants of type-2 GDAs however showed no statistically significant recovery of Grid Walk performance compared to untreated controls at all time points studied (FIG. 15). This is the first demonstration of a difference in functional outcome associated with transplantation of different types of astrocytes to the adult central nervous system.

Those skilled in the art will recognize, or be able to ascertain using no more than routine experimentation, many equivalents to the specific embodiments of the method and compositions described herein. Such equivalents are intended to be encompassed by the following claims.

We claim:

1. An isolated population of cells comprising at least 90% GDAs.

2. The isolated population of claim 1 comprising at least 95% GDAs.

3. The isolated population of claim 1 comprising at least 99% GDAs.

4. The isolated population of claim 1, wherein the population comprises less than 10% type-2 GDAs.

5. The isolated population of claim 1, wherein the population comprises less than 5% type-2 GDAs.

6. A composition comprising the isolated population of claim 1 and a culture medium or a pharmaceutically acceptable carrier.

7. A method of treating a central nervous system (CNS) lesion in a subject, comprising administering to the subject a composition comprising glial restricted precursor (GRP) derived astrocytes (GDAs).

8. The method of claim 7, wherein the GDAs are derived from a GRP by contacting the GRP with a bone morphogenetic protein (BMP).

9. The method of claim 7, wherein the GDAs are GFAP positive and A2B5 negative.

10. The method of claim 7, wherein the GDAs express brain-derived neurotrophic factor (BDNF).

11. The method of claim 7, wherein at least 10³-10⁸ GDAs are administered to the subject.

12. The method of claim 7, wherein the GDAs promote axon regeneration, suppress astrogliosis, re-align host tissues, delay expression of axon growth inhibitory proteoglycans, or a combination thereof.

13. The method of claim 7, wherein the GDAs are made by the method comprising,
a. isolating subventricular zone cells from the subject,
b. purifying A2B5 positive GRPs, and
c. contacting the A2B5 positive GRPs with a BMP.

14. The method of claim 7, wherein the GDAs are generated by the method comprising,
a. contacting pluripotential or multipotential cells with a molecule that induces differentiation,
b. purifying A2B5 positive GRPs from the cells, and
c. contacting the A2B5 positive GRPs with a BMP.

15. The method of claim 14, wherein the multipotential cells are neuroepithelial stem cells.

16. The method of claim 14, wherein the pluripotential cells are embryonic stem cells.

17. The method of claim 7, wherein the composition further comprises a neurotrophic factor.

18. The method of claim 17, where the neurotrophic factor is selected from the group consisting of nerve growth factor (NGP), glial-derived neurotrophic factor (GDNF), cilliary neurotrophic factor (CNTF), neurotrophic factor-3, neurotrophic factor-4/5, fibroblastic growth factor (FGF), and brain-derived neurotrophic factor (BDNF).

19. The method of claim 7, wherein the composition further comprises an immuno-suppressant.

20. The method of claim 19, wherein the immuno-suppressant is selected from the group consisting of cyclosporin, tacrolimus (FK505), cyclophosamid, azathioprines, methotrexate and mizoribin.

21. The method of claim 8 wherein the BMP is BMP-4.
